(19) United States
(12) Patent Application Publication
(10) Pub. No.: US 2009/0214580 A1
(43) Pub. Date: Aug. 27, 2009

(54) Peptide mixtures with immunomodulatory activity

(76) Inventors: Richard A. Houghten, Solana Beach, CA (US); Clemencia Pinilla, Cardiff, CA (US); Roland Martin, Hamburg (DE); Claus-Steffen Sturzebecher, Berlin (DE); Jacquline Shukaiak-Quant, Rockville, MD (US); Henry F. McFarland, Gaithersburg, MD (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/587,987

(22) PCT Filed: Feb. 2, 2005
(86) PCT No.: PCT/US2005/002962

§ 371 Date: Dec. 2, 2008

Related U.S. Application Data
(60) Provisional application No. 60/541,397, filed on Feb. 2, 2004.

(51) Int. Cl. A61K 39/00; C12N 5/02; A61K 38/16; A61K 38/04; A61K 38/08; A61K 38/10; A61K 38/07

(52) U.S. Cl. 424/185.1; 435/375; 514/12; 514/13; 514/14; 514/15; 514/16; 514/17

(57) ABSTRACT

The present invention is related to complex peptide mixtures with immunomodulatory effects and methods for their use. The invention is further directed toward treatments of diseases through the modulation of the immune system with complex peptide mixtures. The invention is also directed to methods for improving the characteristics of complex peptide mixtures.

### RELATED APPLICATIONS

The present application claims priority to U.S. Provisional Patent Application Ser. No. 60/541,397 filed Feb. 2, 2004, which is herein incorporated by reference in its entirety.

### BACKGROUND OF THE INVENTION

1. Field of the Invention

The present invention relates to peptide mixtures with immunomodulatory activity, for use in clinical and experimental settings.

2. Description of the Related Art

Multiple sclerosis (MS) is a chronic inflammatory disease of the central nervous system (CNS) characterized by extensive mononuclear cell infiltration and demyelination. The end result is damage to myelin and to oligodendrocytes, with varying degrees of axonal destruction and the consequence of neurologic impairment (Martin et al., 1992; Noseworthy et al., 2000). While the exact cause of MS is unknown, it is considered an autoimmune disease based on the examination of lesions showing scattered inflammatory infiltrates (particularly mononuclear cells) (Lassmann et al., 1991; Raine, 1994), the positive therapeutic results after immune modulation or immunosuppression (Hohlfeld, 1997; Bashir and Whitaker, 1998), and the genetic association of disease to the major histocompatability complex (MHC; HLA in humans) (Ebers et al., 1996; Haines et al., 1996; Sawcer et al., 1996). Further evidence stems from the striking similarities between disease in humans and the animal model of MS, Experimental Autoimmune Encephalomyelitis (EAE) (Zamvil and Steinman, 1990; Wekerle et al., 1994), which can be induced in several species following immunization with myelin proteins. The T cell-mediated response that ensues and can be passively transferred to naive animals strongly implicates the autoantigen-specific T cells in EAE, as well in MS.

Glatiramer acetate (GA)/Copolymer-1 is a synthetic random sequence polypeptide comprised of alanine (A), lysine (K), glutamate (E) and tyrosine (Y) at a molar A/K/E/Y ratio of 4.5:3.6:1.5:1 with an average length of 40 to 100 aa (Teitelbaum et al., 1971). Developed over thirty years ago to mimic encephalitogenic components of MBP, it instead inhibited disease in rodents and primates (Teitelbaum et al., 1974; Teitelbaum et al., 1996), and more recently in clinical trials it was found to reduce the rate of exacerbations and decrease numbers of new lesions in relapsing remitting MS (RRMS) patients (Johnson et al., 1995; Johnson et al., 1998). It is speculated that GA interacts with immunomodulatory T cells to suppress the inflammatory immune responses underlying MS.

GA has been shown to ameliorate EAE and reduce the relapse rate in MS by about 30%, which led to its approval as a treatment for relapsing-remitting MS (Teitelbaum et al., 1971; Teitelbaum et al., 1974; Johnson et al., 1995; Johnson et al., 1998; Johnson et al., 2000). GA has been shown to be safe and somewhat effective, although a large percentage of MS patients are nonresponders. However, there exists a need for multiple sclerosis treatments with greater strength and efficacy. In addition, it would be desirable to expand the therapeutic scope of those treatments to treat a greater percentage of patients.

### SUMMARY OF THE INVENTION

The present invention is directed to complex peptide mixtures with biological activity and methods for their use. Accordingly, in one aspect of the invention, a complex mixture comprising a plurality of peptides having a length within the range of 8 to 20 amino acids is provided, such that the peptides can be 8, 9, 10, 11, 12, 13, 14, 15, 16, 17, 18, 19, or 20 residues long, wherein said mixture comprises peptides having a degree of diversity at defined positions in the peptide chain, wherein the degree of diversity in at least one defined position is different from the degree of diversity at least one other defined position, and wherein in a majority of the mixture, the peptides include in at least four positions all of A, E, K, and Y and no other amino acids. Advantageously, the complex mixture contains a majority of peptides that include each of one or two amino acids and no other amino acids in at least one position. Preferably, complex peptide mixtures contain all of A, E, K and Y in at least four positions in 75%, 90%, 95% or substantially all of the peptides in a mixture. More advantageously, peptide mixtures contain all of A, E, K and Y in at least four positions in a majority of the peptides in a mixture, where A, E, K and Y are found in approximately the relative proportion of 6 to 2 to 5 to 1. Preferably, at least one position in a majority of peptides in the mixture includes all of the amino acids in any of the following groups and no others: H, R, and K; and K, H, R and V. It is preferred that the peptide mixtures reduce the cellular immune response in at least one autoimmune disease, such as MS.

Another embodiment of the invention features a complex peptide mixture comprising a plurality of peptides having a length within the range of 8 to 20 amino acids, such that the peptides can be 8, 9, 10, 11, 12, 13, 14, 15, 16, 17, 18, 19, or 20 residues long, wherein said mixture comprises peptides having a degree of diversity at defined positions in the peptide chain and wherein in at least a majority of the mixture, the amino acids in the peptides include the following specified amino acids and no other amino acids: A, E, K and Y in at least four positions; I, L and V in at least one position; H, R and K in at least one position; and P and I in at least one position. Advantageously, position P1 is the N-terminal residue. More advantageously, the N-terminal residue is acetylated. Preferably, A, E, K and Y are present in at least four of positions P1, P2, P3, P4, P6, and (if present), P9. More preferably, the peptides have a length of at least 10 amino acids and A, E, K, and Y are present in all of positions P1, P2, P3, P4, P6, and P9. In some embodiments, in at least a majority of the mixture, the amino acids in the peptides include all of K, H, R, and V and no other amino acids in at least one position of the peptides. In some embodiments, in at least a majority of the mixture, the amino acids at the carboxy terminus of the peptides include both P and I and no other amino acids. Preferably, a majority of the peptides in the mixture have, in at least one position, the same amino acid. More preferably, the N-terminal amino acid is acetylated.

An additional embodiment of the invention includes a method for treating a disease, comprising administering to a vertebrate a complex peptide mixture, wherein said mixture comprises peptides having a constrained degree of diversity at each of at least 4 defined positions in the peptide chain and wherein the degree of diversity in at least one defined position is different from the degree of diversity in at least one other defined position. Preferably, the peptides in said mixture are from about 8 to about 20 amino acids in length and the N-terminal peptide, designated as position P1, is preferably acetylated. In some embodiments, in at least a majority of the mixture, the amino acids in the peptides include all of the following specified amino acids and no other amino acids: A, E, K, and Y in at least four positions; I, L, and V in at least one position; and H, R, and K in at least one position. In some embodiments, in at least a majority of the mixture, the amino acids in the peptides include all of the following specified amino acids and no other amino acids in at least one position: K, H, R, and V. Preferably, in at least a majority of the mixture, the amino acids at the carboxy terminus of the peptides include both P and I and no other amino acids. Advantageously, a majority of the peptides in the mixture have, in at least one position, the same amino acid. More advantageously, substantially all of the peptides in the mixture have, in at least one position, the same amino acid. In some embodiments, the method is directed toward the treatment of an autoimmune condition by reducing the T-cell response to self-antigen. Preferably, the method comprises treatment of multiple sclerosis or experimental autoimmune encephalomyelitis.

In one embodiment, a method for stimulating an immune cell is provided, comprising administering to said cell a complex peptide mixture, wherein said mixture comprises peptides having a constrained degree of diversity at each of at least 4 defined positions in the peptide chain, and wherein the degree of diversity in at least one defined position is different from the degree of diversity in at least one other defined position. In some embodiments, the constrained degree of diversity is created by limiting the possible amino acids at each of the said defined positions to a defined list of amino acids that includes less than 10 amino acids. In some embodiments, the stimulating of an immune cell occurs in vivo. Preferably, the administering step is administration by intravenous delivery, intramuscular delivery, delivery via the gastrointestinal tract or transdermal delivery.

Another embodiment features a method for suppression of an immune reaction to an antigen, comprising the administration of a peptide complex mixture to an individual, said complex mixture comprising peptides having a constrained degree of diversity at each of at least 10 defined positions in the peptide chain, and wherein the degree of diversity in at least one defined position is different from the degree of diversity in at least one other defined position. In some embodiments, the immune reaction is an autoimmune reaction. Preferably, the antigen is derived from myelin. Advantageously, the peptide complex mixture comprises peptides of a defined length and formula comprising at least one position with a specific amino acid and at least one position with two to six possible amino acids. In some embodiments, the peptide complex mixture is derived from deconvolution of a complex mixture with more diversity. In particular embodiments, the complex mixture with more diversity is a mixture whose formula is selected from the group consisting of the formulas listed in Table 1. Preferably, the activity of said peptide complex mixture is evaluated by an assay selected from the group consisting of a proliferation assay, a cytokine assay and a ⁵¹Cr release assay. In some embodiments, the suppression of an autoimmune reaction to myelin proteins is due to a mechanism selected from the group consisting of MHC blockade, TCR antagonism, tolerance induction, immune deviation/bystander suppression and cross reactivity with an antigen derived from a protein expressed within the central nervous system. Preferably, myelin antigen is derived from a protein selected from the group consisting of myelin basic protein, proteolipid protein and myelin oligodendrocyte glycoprotein. Advantageously, the antigen is derived from a protein expressed by a cell found in the central nervous system.

An additional embodiment provides a method of inducing an anti-inflammatory response from immune system cells, comprising the administration of a peptide complex mixture to immune system cells, said complex mixture comprising peptides having a constrained degree of diversity at each of at least 10 defined positions in the peptide chain, and wherein the degree of diversity in at least one defined position is different from the degree of diversity in at least another defined position. Preferably, the anti-inflammatory response comprises an alteration in immune cell activity selected from the group consisting of an upregulation of Th2/Th3 cell activity and a downregulation of Th1 cell activity. More preferably, the anti-inflammatory response comprises an increase in the release of cytokines by cells, said cytokines selected from the group consisting of IL-4, IL-5, IL-10, TGF-beta and IL-13. In some embodiments, the inducing of an anti-inflammatory response occurs in vivo. Advantageously, the administration is performed by intravenous delivery, intramuscular delivery, delivery via the gastrointestinal tract or transdermal delivery.

Another embodiment is a method for creating a high affinity peptide ligand of a defined formula for a receptor of a T cell that is reactive to a myelin antigen, comprising the deconvolution of a peptide complex mixture of a formula selected from the group consisting of the formulas listed in Table 1.

In an additional embodiment, a complex peptide mixture is provided, wherein said mixture comprises peptides between 4 and 100 amino acid residues in length, wherein said mixture has a constrained degree of diversity at each of at least 10 defined positions in the peptide chain, and wherein the degree of diversity in at least one defined position is different from the degree of diversity in at least one other defined position. Preferably, the constrained degree of diversity is created by limiting the possible amino acids at each of the said defined positions to a list of possible amino acids that includes less than 10 amino acids. More preferably, the list of possible amino acids for at least two of the said defined positions includes only 1, 2, or 3 amino acids. Advantageously, a majority of the peptides in the mixture have a length between about 4 and about 30 amino acid residues. More advantageously, a majority of the peptides in the mixture vary in length by no more than 4 amino acid residues. In some embodiments, a majority of the peptides in the mixture are acetylated. At least one position of one peptide in the mixture may be occupied by a D-amino acid in additional embodiments.

In yet another embodiment, a method of enhancing a biological property of a complex mixture of peptides, peptidomimetics, or peptides and peptidomimetics is provided, comprising providing a set of complex mixtures of reduced complexity that have a reduced degree of diversity from said complex mixture at least at one position, testing each of said complex mixtures of reduced complexity and said complex mixture in an assay, identifying complex mixtures of reduced complexity that have a greater or lesser activity in said assay than said complex mixture, and enhancing the biological property of said complex mixture by incorporating the complexity-reducing features of at least one of said complex mixtures of reduced complexity that have greater or lesser activity in said assay than said complex mixture into the formula of said complex mixture. Preferably, the biological property is the ability to stimulate an immune system activity or the ability to suppress an immune system activity. In some embodiments, the immune system activity is selected from the group consisting of the clonal expansion of an immune system cell, the differentiation of an immune system cell, the activation of an immune system cell, the creation of an anergic state in an immune system cell, the creation of a memory immune cell population and the secretion of cytokines from an immune system cell. Advantageously, the complex mixtures of reduced complexity comprise mixtures where the identity of the residue or amino acid in one or more positions in the mixture formula is limited to one functionality or amino acid. More advantageously, the complex mixtures of reduced complexity comprise mixtures wherein the identity of the residue or amino acid in one or more positions in the mixture formula is limited to a formula less diverse than the formula for that position in said complex mixture. In some embodiments, the assay is selected from the group consisting of an in vitro assay of peptide recognition by an immune cell population and an assay of the effects of mixture administration on an organism. Preferably, the assay of the effects of mixture administration on an organism is an assay of disease progression in the EAE mouse model. In some embodiments, the enhancing of the biological activity of said complex mixture comprises the limiting of the identity of the residue or amino acid at a position in the compound formula to a single functionality or amino acid. In some embodiments, the biological property is the response measured in an in vivo model of disease, including a model selected from the group comprising a model of anxiety, depression, obesity, cognition, neurological disorder, motor disorder, or pain.

An additional embodiment provides for a complex peptide mixture, comprising a plurality of peptides having a length within the range of 8 to 20 amino acids, wherein said mixture comprises peptides having a degree of diversity at defined positions in the peptide chain, and wherein at least in a majority of the mixture, the identity of 9 contiguous amino acids in the peptides are defined by the formulas FW-EF-EK-AEK-AKY-ANY-ANY-AINV-ASV-Y or EFWY-EFIVWY-EFKQ-AEKQ-AKQY-ANQY-AGNSY-AGINSV-AIQSV-IKRSVY. In some embodiments, position P1 of the 9 contiguous amino acid residues is the N-terminal peptide. Preferably, the N-terminal amino acid is acetylated. Advantageously, a majority of the peptides in the mixture have, in at least one position, the same amino acid. More advantageously, substantially all of the peptides in the mixture have, in at least one position, the same amino acid.

An additional embodiment provides for a complex peptide mixture, comprising a plurality of peptides having a length within the range of 8 to 20 amino acids, such that the peptides can be 8, 9, 10, 11, 12, 13, 14, 15, 16, 17, 18, 19, or 20 residues long, wherein said mixture comprises peptides having a degree of diversity at defined positions in the peptide chain, wherein the degree of diversity in at least one defined position is different from the degree of diversity in at least one other defined position, and wherein in a majority of the mixture, the peptides include in at least one but no more than five positions all of A, E, K, and Y and no other amino acids.

Another embodiment of the invention features a complex peptide mixture comprising a plurality of peptides having a length within the range of 5 to 25 amino acids, such that the peptides can be 5, 6, 7, 8, 9, 10, 11, 12, 13, 14, 15, 16, 17, 18, 19, 20, 21, 22, 23, 24 or 25 residues long, or a combination of one or more of these lengths, wherein said mixture comprises peptides having a degree of diversity at defined positions in the peptide chain. In a further embodiment, P1, P2, P3, P4, P5, P6, P7, P8, P9, and P10 (to the extent they exist) of the peptide mixture is limited to a group of defined amino acids consisting of 1, 2, 3, 4, 5, 6, 7, 8, 9, or 10 amino acids. The groups of defined amino acids can include any combination of the twenty standard amino acids. Preferably, specific groups of amino acids which can be included or excluded from any of defined position include the groups of amino acids: A, E, K, Y; F, L, M, Y; I, M, Q, V; K, R; I, L, V; F, I, L, M, V; F, W; E, F; E, K; A, E, K; A, K, Y; A, N, Y; A, I, N, V; A, S, V; Y; E, F, W, Y; E, F, I, V, W, Y; E, F, K, Q; A, E, K, Q; A, K, Q, Y; A, N, Q, Y; A, G, N, S, Y; A, G, I, N, S, V; A, I, Q, S, V; I, K, R, S, V, Y; K, H, R, V; H, K, R; P, I; A; E; K; A, E; A, K; A, Y; E, Y; K, Y; A, E, Y; and E, K, Y.

In any of the embodiments, the peptides in the complex mixtures can be as few as two amino acids in length to as many as about 100 amino acids in length. The lower limit of the range of lengths can be about 2, 5, 10, 15, 20, 25, 30, 35, 40, 45, 50, 55, 60, 65, 70, 75, 80, 85, 90, or 95 amino acids. The upper limit of the range of lengths can be about 5, 10, 15, 20, 25, 30, 35, 40, 45, 50, 55, 60, 65, 70, 75, 80, 85, 90, 95, or 100 amino acids. More preferably, the length can range from about 5 to about 100, or more preferably from about 5 to about 90, or more preferably from about 5 to about 80, or more preferably from about 5 to about 70, or more preferably from about 5 to about 60, or more preferably from about 5 to about 50, or more preferably from about 5 to about 40, or more preferably from about 5 to about 35, or more preferably from about 5 to about 30, or more preferably from about 5 to about 25, or more preferably from about 8 to about 20. Other ranges of lengths include from about 10 to about 100, or more preferably from about 10 to about 90, or more preferably from about 10 to about 80, or more preferably from about 10 to about 70, or more preferably from about 10 to about 60, or more preferably from about 10 to about 50, or more preferably from about 10 to about 40, or more preferably from about 10 to about 30, or more preferably from about 10 to about 20. Other ranges of lengths include from about 20 to about 100, or more preferably from about 20 to about 90, or more preferably from about 20 to about 80, or more preferably from about 20 to about 70, or more preferably from about 20 to about 60, or more preferably from about 20 to about 50, or more preferably from about 20 to about 40, or more preferably from about 20 to about 30. As used herein, “about” refers to a difference of a few amino acids, for example 1, 2, 3, 4, or 5 amino acids.

In any of the embodiments, the peptide can be modified to created one or more peptidomimetics, such that the complex peptide mixtures contains some portion of peptidomimetics. The portion of the complex mixture which is a peptidomimetic can be 50%, 55%, 65%, 70%, 75%, 80%, 85%, 90%, 95%, 99% or substantially all of the complex mixture.

### BRIEF DESCRIPTION OF THE DRAWINGS

FIG. 1: Peptide length and composition affect the stimulatory potential of complex mixtures (CM). The stimulatory capacity of CM ranging in size and composition were tested in PBMC from nine healthy donors in both primary (A,C) and secondary (B,D) proliferations to confirm the specificity of the response. 20 μg/ml of CM or GA were each added to 18 wells and the % of positive wells (those wells with CPM having an SI greater than twice that observed in background/control wells) determined from pooled data from all donors. Figures A and B analyze the contribution of peptide length and Figures C and D the composition or MHC-bias to stimulatory potential.

FIG. 2: Proliferative responses of MS patient PBMC to CM and GA. The stimulatory capacity of CM with varying lengths and compositions were tested in (A) primary proliferations and (B) secondary proliferations to confirm the specificity of the response. PBMC from 5 MS patients were seeded and each CM tested in 18 wells per donor. Data from the five patients were pooled and reported as the % of total positive wells having CPM at an SI greater than 2 compared to background counts.

FIG. 3: SJL mouse spleen primary proliferative responses to CM. Mouse splenic cells were seeded with 3 different doses of CM (40, 10 and 2.5 μg/ml) or GA to test for stimulatory potential of CM of (A) varying length or (B) differential anchor bias. 18 wells were seeded per antigen, and data from 2 experiments were pooled. % positive wells represents the proportion of wells with CPM at a SI greater than 2 compared to background/control wells.

FIG. 4. Cross reactivity of CM-specific lines to MBP. SJL mice were immunized with CM or GA to enhance the number of CM-specific T cells to assess for cross-reactivity to MBP. Spleen cells were removed from mice ten days post immunization and set into culture (6 wells per condition) with the initial immunizing antigen or human MBP at the μg/ml concentrations shown on the X axes. *p<0.05 by Student's t test comparing proliferation in test wells to control wells without antigen.

FIG. 5: CM reduce clinical severity in PLP139-151-induced EAE. SJL mice were co-injected subcutaneously with 75 μg of PLP 139-151 peptide and 500 μg of the CM, GA, or PLP alone. Progression of disease was blindly monitored for the appearance of clinical symptoms. Results represent the mean daily score. At least five mice per group were used in each of two independent identical experiments that were combined. Only those CM found to significantly alter the severity of disease compared to PLP alone in either the initial disease course or during subsequent relapse are shown.

### DETAILED DESCRIPTION OF THE PREFERRED EMBODIMENT

Approaches to treatment of immunological disorders have changed as understanding of the underlining mechanisms of disease has improved. Traditional treatments of autoimmune diseases such as multiple sclerosis and rheumatoid arthritis include powerful anti-inflammatory steroid compounds and immunosuppressive agents. These treatments have systemic effects and cause severe and untenable side effects. With advances in the understanding of immune cell populations, their maturation and signaling over the last decade, newer treatment for suppression of autoimmune disease that specifically target the cellular mediators of disease have been envisioned.

Cellular immune studies in both the EAE model and in MS patients showed that autoreactive T cells are directed against a limited number of specific peptides such as the “immunomodulatory” domain of myelin basic protein (MBP), MBP (83-99) (Ota et al., 1990; Martin et al., 1991). Historically, it was believed that these autoreactive T cells, but also T cells in general, are exquisitely specific for a few antigens or even single peptides. It has become clear from both theoretical considerations (Mason, 1998) and elegant studies that dissected the fine specificity of T cell clones (TCC), that recognition of peptide/MHC complexes by single T cell receptors (TCRs) is broader than originally thought (Allen, 1994; Ashton-Rickardt and Tonegawa, 1994; Kersh and Allen, 1996; Germain and Stefanova, 1999). These observations led not only to a better understanding of thymic selection processes in that they explained how self peptides that are recognized with intermediate affinity stimulate positive selection of the responding thymocytes, but furthermore explained how peptides with alterations in single or multiple positions, i.e. altered peptide ligands (APL), can elicit a broad range of functional responses in mature T cells and create TCR agonists, partial agonists or antagonists. Studies of MBP (87-99)-specific TCC using positional scanning combinatorial libraries (PSCL) allowed the identification of numerous viral epitopes as well as stimulatory peptides entirely different from those used initially for selection (Hemmer et al., 1997). Interestingly, in the case of autoreactive TCC, this methodology led to the systematic demonstration of antigen peptides with much higher stimulatory potential, i.e. superagonists (Hemmer et al., 2000). Collectively, these observations have important implications not only for the activation of potentially autoreactive T cell clones by foreign agents, e.g. via molecular mimicry, but are probably even more relevant for maintaining the peripheral T cell repertoire.

As indicated above, APL were developed as partial agonists or antagonists in order to modify T cell effector functions (proliferation, cytokine production, apoptosis) (Kersh and Allen, 1996; Madrenas et al., 1996; Germain and Stefanova, 1999) or to induce bystander suppression. The latter mechanism refers to the generation of an APL-specific T cell population that is cross-reactive with the native antigen, e.g. MBP (83-99), but secretes immunomodulatory cytokines such as IL-4 or IL-13 in the case of Th1-mediated autoimmune diseases. These T cell populations are able to downmodulate inflammatory activity when the native autoantigen is released. In vivo studies in rodents and in vitro in MS patients showed that weaker signals initiated by such APL resulted in cytokine bias to a Th2 immunomodulatory profile, and also limited disease in rodent EAE (Windhagen et al., 1995; Brocke et al., 1996; Ausubel et al., 1997; Nicholson et al., 1997; Crowe et al., 2000). However, clinical trials using a MBP (83-99)-based APL confirmed that APLs could also serve as effective self-antigens and were terminated when APL treatment activated autoreactive MBP-specific T cells and caused a flare-up of disease in some patients and in anothers led to APL-induced systemic hypersensitivity (Bielekova et al., 2000; Kappos et al., 2000). This raised the concern that it may be difficult to avoid the generation of further cross-reactive and thus potentially pathogenic autoreactive populations using a single APL for antigen-specific immunotherapy. Combined with the extensive heterogeneity of MS and likely involvement of numerous autoantigens in the course of disease (epitope spreading observed in mice, though less clearly of relevance in MS patients), the therapeutic potential of single APL therapies appears limited.

GA is a random co-polymer composed of alanine, lysine, glutamate and tyrosine in a particular ratio, with an average molecular size between 40 and 100 amino acids. While the precise mechanism of GA immunomodulation is still not fully characterized, it is most likely a combination of MHC blockade, TCR antagonism, tolerance induction, immune deviation/bystander suppression and cross reactivity with MBP. Each has been suggested in humans from in vitro studies and demonstrated in vivo in animal studies (Aharoni et al., 1997, 1998; Miller et al., 1998; Aharoni et al., 2000; Duda et al., 2000; Gran et al., 2000; Neuhaus et al., 2001). Efficient binding to MHC class II molecules (DR in humans and IA in rodents) are postulated to limit autoantigen-MHC interactions (Racke et al., 1992; Fridkis-Hareli et al., 1999) and/or lead to a Th2-type T cell response with resultant bystander suppression of the pathogenic Th1-type response and thus modification of disease in vivo (Duda et al., 2000; Gran et al., 2000; Neuhaus et al., 2000; Chen et al., 2001).

The properties of GA that allow its effects on the immune system have not been fully elucidated. The randomness and complexity of GA are probably responsible for the broad range of T cells that are stimulated via cross reactivity. The composition and size of GA peptides are also likely to be important factors for the co-polymer's activity. The incorporation of similar amino acids in a similar ratio to those found in MBP leads to T cell cross-reactivity with this myelin antigen. At the same time, the random nature of the co-polymer's sequence likely provides a margin of safety as compared to the use of singular defined APLs, since repetitive immunization with a single peptide species is unlikely to occur with co-polymer usage. The amino acids alanine, glutamic acid, lysine and tyrosine serve as key anchor residues for multiple HLA-DR alleles and are the reason for the strong MHC binding of GA peptides, which can occur in multiple binding registers due to the repetitive presence of these amino acids in the GA polypeptide chain. The net results of these properties of GA are a high degree of stimulation of a large number of T cells, high binding affinities and the displacement of antigens from the MHC site. Additionally, the large size of GA as compared to typical MHC peptide ligands may lead to MHC clustering and thus would interfere with TCR recognition and signaling in response to autoantigens. These unique properties render GA effective and safe; however, the nature of the GA mixture also complicates the study of its mechanisms systematically.

Several studies have attempted to improve GA with the development of new co-polymers while trying to understand the immunomodulatory mechanisms at hand. Initial experiments determined that virtually all of the large variety of copolymers found in the random mixture of poly-YEAK were capable of binding to purified class II MHC proteins, suggesting that a given polypeptide was no more effective or less than the next (Fridkis-Hareli et al., 1999). GA could effectively compete for binding of MBP (85-99) to HLA-DR2 and inhibit DR2-restricted responses to MBP (85-99), despite the fact that structurally the P1 pocket in DR2 is too small to accommodate the hydrophobic tyrosine (Y) residue (Krieger et al., 1991). The latter conclusion does not, however, take into account that the P4 position of DR2b accommodates the bulky tyrosine easily, while the other DR2 allele from the DR15 haplotype, DR2a, will even favor Y in the P1 pocket position. Subsequently, some random copolymers of three amino acids were found to bind with higher affinities to distinct DR molecules than other molecules. For example poly-YEAK and poly-EAK were better binders of DR2 molecules than the other three amino acid copolymers (poly-YEA, -YEK and -YAK) (Fridkis-Hareli et al., 1999).

Limiting the complexity of GA would allow an examination of individual properties of GA and their contribution to GA's immunomodulatory effects. Investigators made efforts to limit the complexity of GA by resorting to smaller molecules and/or improving efficacy by designing copolymers based on residues found in specific immunodominant T cell epitopes interacting with disease-associated MHC molecules. Based on interactions of MBP (85-99) with the HLA-DR molecule HLA-DRB1*1501, novel random four-amino acid copolymers of 14, 35 or 50 a.a. in length were created with the introduction of F (phenylalanine) in place of glutamic acid (E) (Fridkis-Hareli et al., 2002). Both poly-FAK and poly-FEAK were more effective than GA in the inhibition of MBP(85-99)-specific HLA-DR2-restricted TCC activation. Poly-FAK and -FEAK also suppressed EAE in the SJL mouse strain more efficiently than GA. Another study synthesized peptides based on the motifs recently described for binding of MBP(85-99) and of GA to the groove of HLA-DR2 molecules, i.e. E at P2, K at P1, Y and A at P1, and A at P2-P11. Certain 15-mer peptides did indeed inhibit binding of MBP (85-99) to HLA-DR2 molecules more effectively than GA. Another report described ordered peptides of repetitive 4 amino acid (aa) sequences designed to bind critical MHC pockets and to interfere with T cell activation, based on MHC-TCR binding motifs for HLA-DR2 and MBP₈₅₋₉₉ (Ruiz et al., 2001). One such sequence, EYYKEYYKEYYK was found to ameliorate EAE in Lewis rats. However, while it appears that synthetic copolymers tailored according to binding motifs of immunodominant epitopes and binding pockets of DR molecules can illuminate aspects of peptide interactions with MHC and TCR, use of single APLs has proven problematic in human clinical trials for MS treatment, as noted above (Bielekova et al., 2000; Kappos et al., 2000).

We have designed peptidic complex mixtures (CM) of defined lengths containing defined amino acid formulas and residues in particular positions. In some embodiments of the invention, the definition of the formulas and residues for particular positions has been guided by published class II binding motifs (Vogt et al., 1994; Kalbus et al., 2001). These motifs indicate which amino acids at particular positions in the mixture peptides can interact with the specificity pockets of MS- (HLA-DRB1*1501 and HLA-DRB5*0101 or HLA-DR2) and EAE (IA^{s})-related MHC molecules. In some embodiments of the invention, in contrast to completely random mixtures, the size and the complexity of the mixtures were limited by the introduction of key amino acids in defined positions. Some embodiments of the invention featured complex mixtures that were designed to study the influence of peptide acetylation, length and MHC anchor bias, as well as the contribution of amino acids found stimulatory to a GA-specific TCC, based on position scanning combinatorial library (PSCL) assays. Several mixtures were created with significant stimulatory potential in PBMC from humans or mouse spleen cells, suggesting a high degree of cross-reactivity with other peptide antigens. A subset of these mixtures exhibited cross reactivity to myelin antigens and prophylactic efficacy in reducing the severity of EAE.

Aspects of the invention involve complex peptide mixtures of a defined length, such as 4, 5, 6, 7, 8, 9, 10, 11, 12, 13, 14, 15, 16, 17, 18, 19 or 20 amino acids in length, or a combination of peptides comprising two or more of these lengths. Some mixtures may contain a majority of peptides of a particular length, with the rest of the mixture comprising peptides of a different length, or peptides of two or more different lengths. In a particular aspect of the invention, shorter complex peptide mixtures were created in order to test the ability of a less complex, and more structurally defined mixture to exert both stimulatory and possibly therapeutic properties relevant to autoimmune processes. Mixtures of limited complexity were developed to study how subtle changes in basic factors including peptide length, structure or composition can alter the stimulatory and therapeutic potential of the mixtures. In some aspects of the invention, complex peptide mixtures were created wherein mixture peptides were acetylated. In particular aspects of the invention, the acetylation was achieved at the N-terminal end of a majority (or preferably, at least about 70%, 80%, 90%) of peptides in the complex mixtures. In other embodiments, the acetylation was achieved at the N-terminal in substantially all of the peptides found in a complex mixture. In additional embodiments, peptides in the mixtures contain acetylated amino acid residues that are not located at the N-terminus.

Complex peptide mixtures may be described with formulas, which can indicate which amino acids can be found in positions relative to other positions. A particular position in a formula may be limited to any subset of desired amino acids, including a single amino acid. If a particular position is limited to more than one amino acid, the amino acids available to occupy the position in one embodiment may be substantially equally available. In some embodiments, an amino acid that may occupy a particular position in the peptides of a mixture is found in substantially the same number of mixture peptides as the other amino acids that may occupy the position. In some embodiments, there may be a ratio to the amount of each possible amino acid found in a position among the peptides of a mixture. In some embodiments where more than one amino acid can be found in a particular position in the mixture peptides, there may be an unequal number of peptides containing one particular amino acid in that position as compared to peptides containing a different amino acid in that position. The sequence of amino acids in a formula may begin at the N-terminal end of a majority of peptides in a mixture. In additional embodiments of the invention, the sequence of the amino acids in a formula may be contained within the overall peptidic sequence in a majority of the peptides of a mixture (i.e., there exists additional amino acid residues both N-terminal and C-terminal to the residues of the formula), or may be found at the N-terminal end or the C-terminal end of the peptidic sequence in a majority of the peptides in a mixture. The formulas of the invention may be manipulated to alter the diversity of the formula. In some aspects of the invention, formulas contain the same group of possible amino acids for each position in the formula. In other aspects of the invention, formulas contain at least one position where the identity of the possible amino acids for that position differ from the identity of the possible amino acids in at least one other position of the formula. Preferably, there are at least 3 positions, and optionally 4, 5, 6, 7, 8, or more positions in which the identity of possible amino acids is not the same. In some embodiments, the identity of the possible amino acids in two or three or more positions is the same (preferably a constrained set), but the possible amino acids at those positions differ from the set of possible amino acids at the rest of the positions in the peptides mixture.

In some embodiments of the invention, 3, 4, 5, 6, 7, or more positions in the formulas are constrained to include less than 19 possible amino acids (excluding cysteine), and preferably less than 10, 8, 7, 6, or even 5 amino acids. Similarly, in some embodiments, 2, 3, 4, 5, 6, or more positions of the formulas are limited to between 1 and 4 amino acids, preferably wherein the set of possible amino acids at least at 1 or 2 of such positions is different from the possible amino acids at any of the other such positions.

In some embodiments of the invention, a majority of the peptides in a complex mixture contain an amino acid sequence that conforms to a formula of the invention. In additional embodiments of the invention, 75%, 80%, 85%, 90%, 95%, 99%, substantially all, or some other proportion of the peptides in a complex mixture contain an amino acid sequence that conforms to a formula of the invention.

In preferred embodiments, the complex mixture is an immunomodulatory mixture, and has a measurable effect on a mammalian immune response to at least one antigen, preferably to a self-antigen.

Formulas of complex peptide mixtures of the invention may specify any amino acid or amino acid analogue. Some embodiments of the invention utilize any of the standard amino acids in a majority of the positions in a formula. Additional embodiments are limited to any of the standard amino acids except for cysteine. Cysteine is not utilized for complex mixture peptide synthesis in some embodiments to prevent the creation of peptide secondary structure. In some embodiments of the invention, the use of non-standard or non-natural amino acids is envisioned; amino acids in altered molecular configurations, such as D-amino acids, and amino acids with non-standard side chains are examples of the types of molecules that may be specified. Like acetylation, use of these analogues of standard amino acids can confer advantageous properties to complex mixture peptides, such as resistance to degradation, improved delivery or compartmentalization and increased immunomodulatory activity.

In some embodiments, the complex mixture contains peptidomimetics. As used herein, peptidomimetic refers to peptide analogs, including the use of one or more amino acid analogs discussed above. While the term “amino acid” is used throughout the specification, where the peptide is modified, it is understood that “amino acid” refers to whatever residue has replaced the amino acid in the peptide. For example, the following modifications of a peptide create a peptidomimetic: one or more amide bonds can be reduced to amines as shown in (1) of Formula I below; one or more amide bonds can be functionalized as shown in (2), wherein R₁ can be any aldehyde, or a carboxylic acid at the free amine; or a heterocycle can be created along the backbone as shown in (3) and (4). These compounds can mimic the activity of the analog peptide while improving some of the limitations of peptides such as degradation. Examples of peptidomimetics and how they can be made can be found in Nefzi, et al., Bioorganic & Medicinal Chemistry Letters, 8:2273-2278 (1998) and Nefzi et al., Journal of Organic Chemistry, 69:3603-3609 (2004), which are both herein incorporated by reference in their entirety.

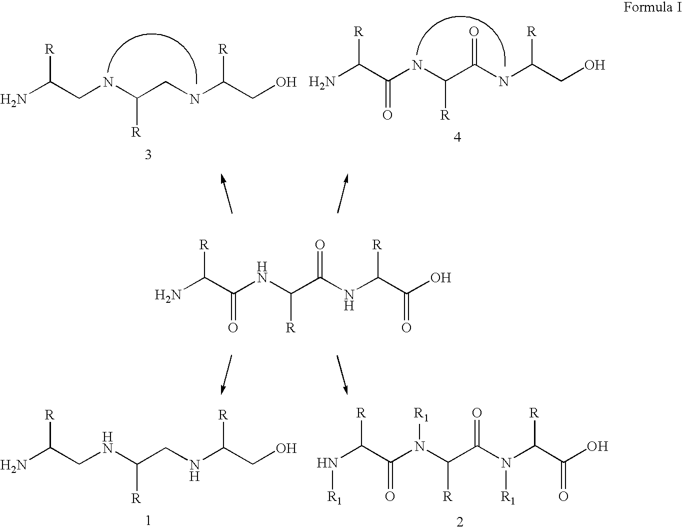

Formulas for complex peptide mixtures may specify that a certain number of possible amino acids can be found at a particular position. The particular position may be an absolute position. For example, in some embodiments, the majority of peptides in a mixture contain one of A, E, K or Y in P4, i.e. the fourth residue from the N-terminal residue. In additional embodiments, the particular specified position is simply relative to other specified positions. For example, a formula may indicate that A, E, K or Y are found in positions P1 and P5 while I, L or V are found in position P6, in a majority of the peptides in a mixture. In this example, P1 may be any residue in a particular peptide except for any of the last 5 residues at the C-terminal end; A, E, K or Y would be also be found four positions toward the C-terminal end from the first position containing A, E, K or Y and I, L or V would be found in the next C-terminal position after the second A, E, K or Y position. In additional embodiments, formulas for complex peptide mixtures may specify that a certain number of possible amino acids can be found in a particular number of positions in the peptides of a mixture, but not specify to which particular positions that this requirement pertains, in terms of relative or absolute position number. For example, a formula may indicate that the majority of the peptides in a mixture contain at least four positions at which the amino acids available for those positions are A, E, K and Y. In these embodiments, a majority of the peptides in a mixture would contain A, E, K or Y in their sequence at least 4 times. Formulas may specify the number of positions containing a particular subset of amino acids, the specific positions containing those subsets, either in terms of absolute position in peptides or in terms of relative position to other positions in the formula, or a combination of any two or all three types of formulaic specifying, for a majority of the peptides in a mixture, substantially all the peptides in a mixture or some fraction thereof. In some embodiments of the invention, one or more formulas are repeated and can be found two or more times within the same peptide in a majority of the peptides of a complex mixture. Additional embodiments feature one or more formulas that are found only once in a peptide in a majority of peptides in a complex mixture, while one or more other formulas are found two or more times in the same peptide in a majority of peptide in a complex mixture. In some embodiments of the invention, formulas designate the particular order of groups of possible amino acids found in substantially all or some fraction of mixture peptides by listing the groups in order, separated by dashes.

Additional embodiments of the invention feature complex mixtures defined by formulas devised through experiments utilizing positional scanning combinatorial libraries (PSCL) and GA-specific T cell clones (GA TCC). The use of PSCL is well-known technique in the art (for a review, see Borras et al., 2002, which is hereby incorporated by reference in its entirety) and can be used to limit the diversity of complex mixtures. For a complex mixture of a particular formula, sublibrary mixtures are created where the identity of the amino acid in one position is fixed for substantially all of the peptides in the mixture. Typically, nineteen sublibrary mixtures are created for each position, corresponding to the fixing of the identity of the amino acid to one of the 19 standard, non-cysteine amino acids in that position. A range of sublibrary mixtures can be created for any number of positions in the complex peptide mixture formula. In some embodiments, all of the sublibrary mixtures have one or more positions wherein the identity of the amino acid at that position is fixed to one particular amino acid. In other embodiments, the identity of the amino acid in a particular position is defined by a formula wherein the amino acid in that position can be selected from two to 18 amino acids. Additional embodiments envision the use of amino acid analogues. Each sublibrary mixture can be tested for activity in an assay. In particular embodiments of the invention, sublibrary mixtures were tested for their ability to activate GA TCCs. In some of these embodiments, the contribution of each amino acid in each position to the overall activity of the parent complex mixture was examined. Several amino acids in a particular position may demonstrate high levels of activity in an assay. Through the use of PSCL, the formula used to create the complex peptide mixture can be altered by eliminating lower activity amino acids from certain positions, leaving only one amino acid or a selection of amino acids showing high levels of activity. A complex peptide mixture that uses the altered formula can be less diverse. In a formula of reduced diversity, created by eliminating lower activity peptides from the mixture, the activity of the mixture per unit of peptide mass can be increased. Some embodiments of the invention are compatible for testing with a wide variety of laboratory assay techniques, both in vivo and in vitro. In particular embodiments, the activities of sublibrary mixtures are measured using assays of in vitro cellular proliferation, cytokine release assays and/or ⁵¹Cr release assays. PSCL techniques can be used to identify the most active amino acid residue for one or more positions in a complex peptide mixture formula. In certain embodiments, PSCL techniques are used to create a defined formula for a single peptide that serves as a high-activity peptide ligand for a T cell population.

In some embodiments of the invention, peptides found in complex peptide mixtures interact with immune system cells through receptor proteins on the surface of the cells. The interactions of complex mixture peptides with immune cell surface receptor proteins may lead to changes in conformation of the receptor protein or some part of the receptor protein, a change in the activation state of the receptor protein or a change in the ability of the receptor protein to interact with other proteins, on the surface of the same cell or on other cells. In some embodiments, the interaction of peptides found in complex peptide mixtures of the invention with immune cell surface receptors reduces or substantially eliminates the ability of the receptor to interact with other peptides. For example, the interaction of peptides and class II MHC molecules on the surface of cells can lead to MHC blockade, where the interaction of the complex mixture peptides with MHC molecules inhibits the activation of T cells by other peptides binding to the same MHC molecules. In some embodiments, the interaction between peptides found in complex mixtures and T cell receptors can lead to T cell antagonism, where the response of T cell receptor to the presentation of normally stimulatory peptides is inhibited. In particular embodiments, the interaction of peptides found in complex peptide mixtures leads to changes in immune system cells on a cellular level. The interaction may stimulate cells to become active, to proliferate and/or to secrete signaling molecules, such as cytokines and interferons. In some embodiments, the interaction of peptides found in complex mixtures with cell surface receptors leads to differentiation of immune cells into more mature types of cells, a memory immune cell population for example. The activity of the immune system cells exposed to peptides of the invention may lead to an increase or a reduction of inflammation. For example, in some embodiments, peptides found in complex peptide mixtures interact with receptors on the surface of Th2- and Th3-type T cells and stimulate proliferation of these cells. The cells then migrate to the central nervous system and are stimulated by self-antigens to secrete anti-inflammatory signalling molecules, such as IL-4. The presence of anti-inflammatory cytokines leads to bystander suppression, where the activity of autoimmune, inflammatory CD4⁺ T cells in the central nervous system is reduced. In other embodiments, the interaction of peptides found in complex peptide mixtures with immune cell receptor proteins leads to a reduction in cellular activity or the creation of an anergic state. In certain circumstances, for example during exposure to particular cytokines, the interaction of peptides found in complex mixtures and cell surface receptors can create long-term T cell tolerance of antigenic peptides. In particular embodiments, the interaction of peptides found in complex peptide mixtures with immune cell surface receptors leads to alterations in the general state of the immune system, such as the reaction of an organism to exposure to a particular antigen. Additional aspects of the invention feature a reduction in autoimmunity as a result of the interaction between peptides found in complex mixtures of the invention and immune cell surface receptor proteins. In certain embodiments, the interaction of peptides and surface receptor proteins reduces the level of an organism's reaction to a self-antigen derived from a protein found in the central nervous system. In particular embodiments, this self-antigen is derived from myelin and the reaction is an autoimmune inflammatory reaction.

In some embodiments of the invention, complex peptide mixtures are administered to vertebrates. The deliver of peptides to vertebrates can occur through one or more of multiple delivery schemes. Intravenous delivery, intramuscular delivery, transdermal delivery and other forms of parenteral delivery are featured in certain embodiments of the invention, as is delivery via the gastrointestinal tract.

Unless defined otherwise, technical and scientific terms used herein have the same meaning as commonly understood by one of ordinary skill in the art to which this invention belongs. One skilled in the art will recognize many methods and materials similar or equivalent to those described herein, which can be used in the practice of the present invention. Indeed, the present invention is in no way limited to the methods and materials described. For purposes of the present invention, some terms are defined below.

“Diversity” refers to the number of different peptides that could be present in a complex mixture of a given formula or constitution. Complex mixtures that can contain a greater number of peptides than another mixture are said to be more diverse. Changes in formulas that define a majority of peptides in a complex mixture can lead to a “constrained degree of diversity” in a complex mixture synthesized according to the new formula. The diversity would be constrained if the number of possible peptides generated by the new formula is less than the number of possible peptides with the old formula. The relative diversity of a complex mixture can also be compared to a random polypeptide mixture wherein each position in each polypeptide can be occupied by any one of 19 standard amino acids (the 20 standard amino acids minus cysteine). The possibility of having any of the 19 standard amino acids in any position in any of the random polypeptide mixture peptides means that this type of mixture has a very high degree of diversity. In addition to comparing the relative diversity of complex mixtures, the theoretical absolute diversity of a complex mixture can be computed by calculating the number of possible peptides from a particular formula for a complex mixture. For example, a complex mixture of tripeptides where each of the three positions in a peptide of the mixture can be occupied by one of four amino acids can contain 43, or 64, possible tripeptides.

“Stimulatory index”, or SI, refers to measurements of the stimulation of immune cell populations in relationship to control populations that are not exposed to the stimulus being tested. SI can be expressed as a number given by the following formula: SI=cpm (Ag)/cpm (without Ag). In certain embodiments of the invention, the activities of TCC stimulated by peptides found in complex mixtures and of control TCC cultures not exposed to peptides are measured. The two measurements are then combined to form the stimulatory index for the peptide mixture to which the experimental T cell population was exposed.

### EXAMPLES

The following examples disclose various applications of the present invention and are not intended to be limiting, but rather illustrative of the present invention.

### Materials and Methods

### Peptides and Reagents:

Complex mixtures (CM) (Table 1, below) and myelin peptides were synthesized as first presented elsewhere by the simultaneous multiple peptide synthesis method, methyl-benzhydrylamine polystyrene resin, and t-Boc-protected L-amino acids (Houghten, R. PNAS 82:5131-5135, and Houghten, R., et al. Journal of Med Chem. 42:3743-3778, 1999, both of which are hereby incorporated by reference in their entireties). Myelin peptide pools: Proteolipid protein (PLP) peptides: PLP₈₉₋₁₀₆-GFYTTGAVRQIFGDYKTT, PLP₁₃₉₋₁₅₄-HCLGKWLGHPDKFVGI, PLP₁₇₈₋₁₉₇-NTWTTCQSIAFPSKTSASIG, PLP₁₉₀₋₂₀₉-SKTSASIGSLCADARMYGVL. Myelin basic protein (MBP) peptides: MBP₁₃₋₃₂-KYLATASTMDHARHGFLPRH, MBP₈₃₋₉₉-ENPVVHFFKNIVTPRTP, MBP₁₁₁₋₁₂₉-LSRFSWGAEGQRPGFGYGG, MBP₁₃₁₋₁₅₅-ASDYKSAHKGLKGVDAQGTLSKIFK, MBP₁₄₆₋₁₇₀-AQGTLSKIFKLGGRDSRSGSP-MARR. Myelin oligodendrocyte glycoprotein (MOG) peptides: MOG₁₋₂₀-CQFRVIGPRHPIRALVGDEV, MOG₁₁₋₃₀-PIRALVGDEVELPCRISPGK, MOG₂₁₋₄₀-ELPCRISPGKNATGMEVGWY, MOG₃₅₋₅₅-MEVGWYRPPFSRVVHLYRNGK. 2′,3′-Cyclic nucleotide 3′phosphodiesterase (CNPase) and myelin oligodendrocytic basic protein (MOBP) peptides: CNPase₃₄₃₋₃₇₃-EVGELSRGKLYSLGNGRWMLTLAKNMEVRAI, CNPase₃₅₆₋₃₈₈-GNGRWMLTLAKNMEVRAIFTGYYGKGKPVPTQG, MOBP₂₁₋₃₉-FSIHCCPPFTFNNSKKEIV and MOBP₃₁₋₄₉-FLNSKKEIVDRKYSICKSG. Peptides were characterized using an electrospray mass spectrophotometer interfaced with a liquid chromatography system. Glatiramer acetate (GA)/Copolymer-1/Copaxone was purchased from Teva Pharmaceuticals, (Teva Marion Partners, Kansas City, Mo.). PLP₁₃₉₋₁₅₁ peptide (sequence HSLGKWLGHPDKF) was synthesized by Stanford Pan Facility (Palo Alto, Calif.). Human MBP was prepared as previously described (Deibler et al., 1972).

TABLE 1
Nomenclature and Composition of Complex Mixtures (CM)

| | | P1 | P2 | P3 | P4 | P5 | P6 | P7 | P8 | P9 | P10 |
|---|---|---|---|---|---|---|---|---|---|---|---|
| 10 mers | | | | | | | | | | | |
| AEKY¹⁰ | | AEKY | AEKY | AEKY | AEKY | AEKY | AEKY | AEKY | AEKY | AEKY | AEKY |
| AEKY¹⁰ | Ac- | AEKY | AEKY | AEKY | AEKY | AEKY | AEKY | AEKY | AEKY | AEKY | AEKY |
| AEKY^{GA}* | | AEKY | AEKY | AEKY | AEKY | AEKY | AEKY | AEKY | AEKY | AEKY | AEKY |
| AEKY^{GA}* | Ac- | AEKY | AEKY | AEKY | AEKY | AEKY | AEKY | AEKY | AEKY | AEKY | AEKY |
| 12 mers | | | | | | | | | | | |
| AEKY¹² | | AEKY | AEKY | AEKY | AEKY | AEKY | AEKY | AEKY | AEKY | AEKY | AEKY . . . |
| AEKY¹² | Ac- | AEKY | AEKY | AEKY | AEKY | AEKY | AEKY | AEKY | AEKY | AEKY | AEKY . . . |
| 15 mers | | | | | | | | | | | |
| AEKY¹⁵ | | AEKY | AEKY | AEKY | AEKY | AEKY | AEKY | AEKY | AEKY | AEKY | AEKY . . . |
| AEKY¹⁵ | Ac- | AEKY | AEKY | AEKY | AEKY | AEKY | AEKY | AEKY | AEKY | AEKY | AEKY . . . |
| 20 mers | | | | | | | | | | | |
| AEKY²⁰ | | AEKY | AEKY | AEKY | AEKY | AEKY | AEKY | AEKY | AEKY | AEKY | AEKY . . . |
| AEKY²⁰ | Ac- | AEKY | AEKY | AEKY | AEKY | AEKY | AEKY | AEKY | AEKY | AEKY | AEKY . . . |
| DR2a bias | | | | | | | | | | | |
| AEKY^{10-DR2a} | | FLMY | AEKY | AEKY | IMQV | AEKY | AEKY | AEKY | AEKY | KR | AEKY |
| AEKY^{10-DR2a} | Ac- | FLMY | AEKY | AEKY | IMQV | AEKY | AEKY | AEKY | AEKY | KR | AEKY |
| DR2b bias | | | | | | | | | | | |
| AEKY^{10-DR2b} | | ILV | AEKY | AEKY | IMQV | AEKY | AEKY | FILMV | AEKY | AEKY | AEKY |
| AEKY^{10-DR2b} | Ac- | ILV | AEKY | AEKY | IMQV | AEKY | AEKY | FILMV | AEKY | AEKY | AEKY |
| GA clone driven | | | | | | | | | | | |
| GA TCC-1¹⁰ | Ac- | FW | EF | EK | AEK | AKY | ANY | ANY | AINV | ASV | Y |
| GA TCC-2¹⁰ | Ac- | EFWY | EFIVWY | EFKQ | AEKQ | AKQY | ANQY | AGNSY | AGINSV | AIQSV | IKRSVY |
| IAs bias | | | | | | | | | | | |
| 19a.a.^{10-IAs} | Ac- | X | X | X | X | KHRV | X | ILV | HRK | X | PI |
| AEKY^{10-IAs} | Ac- | AEKY | AEKY | AEKY | AEKY | KHRV | AEKY | ILV | HRK | AEKY | PI |
| AEKY^{10-IAs-mis} | Ac- | PI | HRK | ILV | KHRV | AEKY | AEKY | AEKY | AEKY | AEKY | AEKY |
| Random amino acids | | | | | | | | | | | |
| 19 a.a.¹⁰ | Ac- | X | X | X | X | X | X | X | X | X | X |

For each CM, the amino acid which could be present at each position in the random peptide mixture is listed in the chart. As shown above, the 10mer AEKY10 could have A, E, K, or Y in each of the 10 peptide positions. Alternatively, DR2a-biased CM AEKY10-DR2a could contain any of the FLMY anchor residues in P1, but no another aa.
*GA indicates that the aa A, E, K and Y were present at ratios similar to that used in GA prior to the synthesis (6:2:5:1), whereas other mixtures contain the listed aa at each position at equimolar ratios.
X represents any of the 19 non-cysteine standard amino acids.

### Patients and Controls:

Peripheral blood mononuclear cells (PBMC) were isolated from five MS patients and nine healthy donors. The patients suffered from relapsing-remitting MS and ranged from 31 to 41 years old. Two patients were on interferon-beta therapy, and the remainder were untreated at the time of blood draw; none had ever received GA. Each patient and six of the healthy donors expressed the MS-associated HLA-DR2 haplotype (HLA-DRB1*1501, HLA-DRB5*0101).

### T Cell Proliferation:

PBMC were isolated from leukocytaphereses and seeded in complete T cell medium (TCM; Iscove's modified Dulbecco's medium [IMDM] containing 2 mM 1-glutamine, 50 μg/ml gentamycin, penicillin-streptomycin 100 U/ml each, and 5% pooled human AB serum) at 2×10⁵ cells/well into 96 well U-bottom microtiter plates (Nunc, Naperville, Ill.) and stimulated with CM and GA at 20 μg/ml. After 6 days, 100 μl samples of cell suspension were removed from these plates and transferred to a new microtiter plate to which 1 μCi/well of H³-thymdidine (Amersham Pharmacia Biotech, Piscataway, N.J.) was added and after 16 hours harvested and counted in a scintillation counter (Microbeta, Wallac, Gatihersburg, Md.). Human recombinant interleukin (IL)-2 (Hoffman-LaRoche, provided by Dr. C. Reynolds, National Cancer Institute, NIH) was added to the original plate at a final concentration of 20 U/ml in fresh TCM and replenished every 2-3 days until the split well-secondary proliferation. After a 2-3 day break from IL-2, 50 μl of the cell suspension was transferred into adjacent wells of a separate 96-well U-bottom plate, and 100 μl of TCM containing 2×10⁵ autologous irradiated (3000 rad) PBMC were added with 50 ml X-vivo media to control wells (no antigen) and 50 μl of CM (20 μg/ml) in X-vivo media. The original plate received irradiated PBMC (as above), antigen at 20 μg/ml and IL-2 (20 U/ml) to maintain the culture. At 48 hours post secondary stimulation plates received thymidine and were harvested and counted 16 hours later as described above. Lines specific for individual CM were generated by subsequent stimulation as for the secondary proliferation, and were tested for cross reactivity with myelin peptide pools (each peptide at 10 μg/ml) in proliferative assays as described above.

### Identification of Key Amino Acids Recognized by a GA-Specific T Cell Clone:

A GA-specific TCC was established from a healthy donor and tested with PSCL to identify stimulatory amino acids in each position of a 10-mer peptide and thus predict antigens for clones of known and unknown specificity. CM were created as described above under “Peptides and reagents” based on two thresholds of stimulatory potential (Table 1, “GA clone driven”): GA TCC-1 included the defined amino acids of the mixtures with stimulatory indices (SIs) 2 times greater than the average SI at each position, and GA TCC-2 included all amino acids with significant stimulatory capacity (SIs greater than 1.2 times the average SI at each position), thus a significantly more complex mixture.

### Immunogenicity of CM in Mice In Vitro:

6-8 week old female SJL/J mice were purchased from Charles River Laboratories (Wilmington, Mass.). Spleen cells were isolated from anesthetized naïve SJL mice or 10 days after immunization with 200 μg of CM or GA in Complete Freund's Adjuvant (CFA—Difco Laboratories, Detroit, Mich.) on four spots on their back and subsequent aseptic spleen removal and red blood cell lysis using ACK lysis buffer from Biosource (Camarillo, Calif.). In primary proliferations, 2×10⁵ cells were seeded in mouse cell media [RPMI 1640 supplemented with 10% fetal calf serum (Gibco BRL, Toronto, ON), non-essential amino acids, penicillin/streptomycin (100 U/ml), sodium pyruvate (1 mM), L-glutamine (2 mM) all from Biowhittaker (Walkersville, Md.)] in the presence of 40, 10 or 2.5 μg/ml of the various CM, PLP, or GA in 96 well round bottom plates (Nunc, Rochester, N.Y.). After six or three days in naïve or immunized mouse experiments respectively, 1 μCi ³[H]-thymidine (Dupont, Wilmington, Del.) was added overnight and subsequently harvested and counted for thymidine incorporation on a Trilux liquid scintillation counter (Wallac, Gaithersburg, Md.).

### Induction of EAE

Animal studies were performed in accordance with NIH animal care and use guidelines. 6-8 week old female SJL mice were immunized with 75 μg PLP in incomplete Freund's adjuvant (IFA) supplemented with Mycobacterium tuberculosis H37RA to 2 mg/ml (both from Difco Laboratories). To test the prophylactic effects of the CM, 500 μg of CM or GA were added into this emulsion prior to immunization. Mice were blindly evaluated for onset of disease and clinical scores were assigned following examination as follows: 0=no clinical signs; 1=flaccid tail; 2=paresis of one or both hindlegs; 3=paralysis of one or both hindlegs; 4=quadriparesis or quadriparalysis; 5=moribund. The cumulative score represents the mean of the summation of single scores recorded in each mouse during the initial disease course (days 0-24) and the remaining disease course (days 24-45).

### Data Analysis:

Proliferative responses of T cell lines to CM or to MBP were compared using ANOVA and Tukey's multiple comparisons to determine significant responses to antigens (p<0.05) compared to unstimulated controls. Cumulative disease scores were compared using ANOVA for significant differences and Students t test to compare scores of CM- or GA-immunized individual mice to mice receiving only PLP during the initial bout of disease and again throughout subsequent relapses.

### Short Complex Mixtures are Stimulatory in Both Humans and Mice

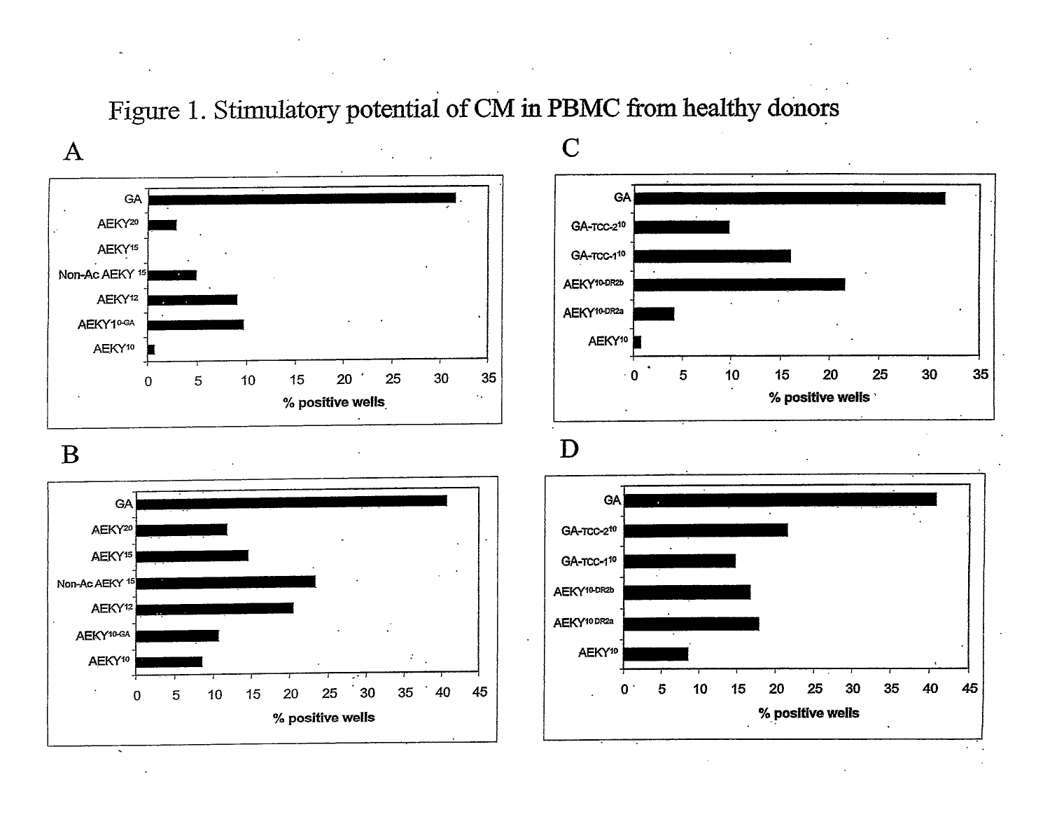

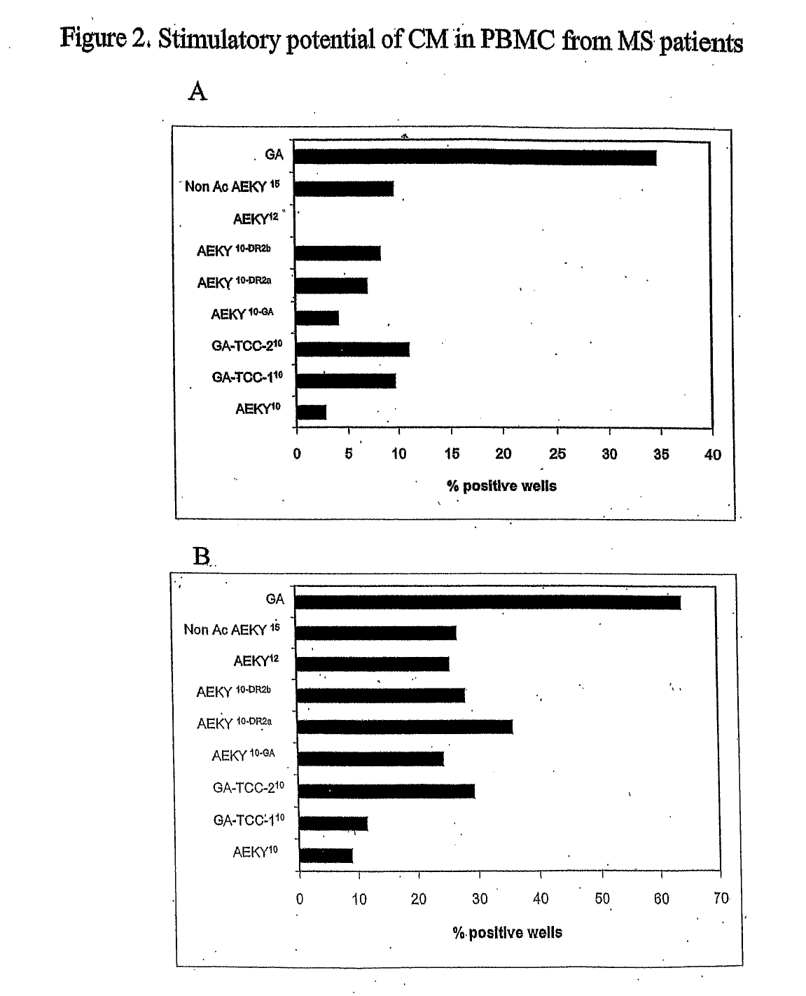

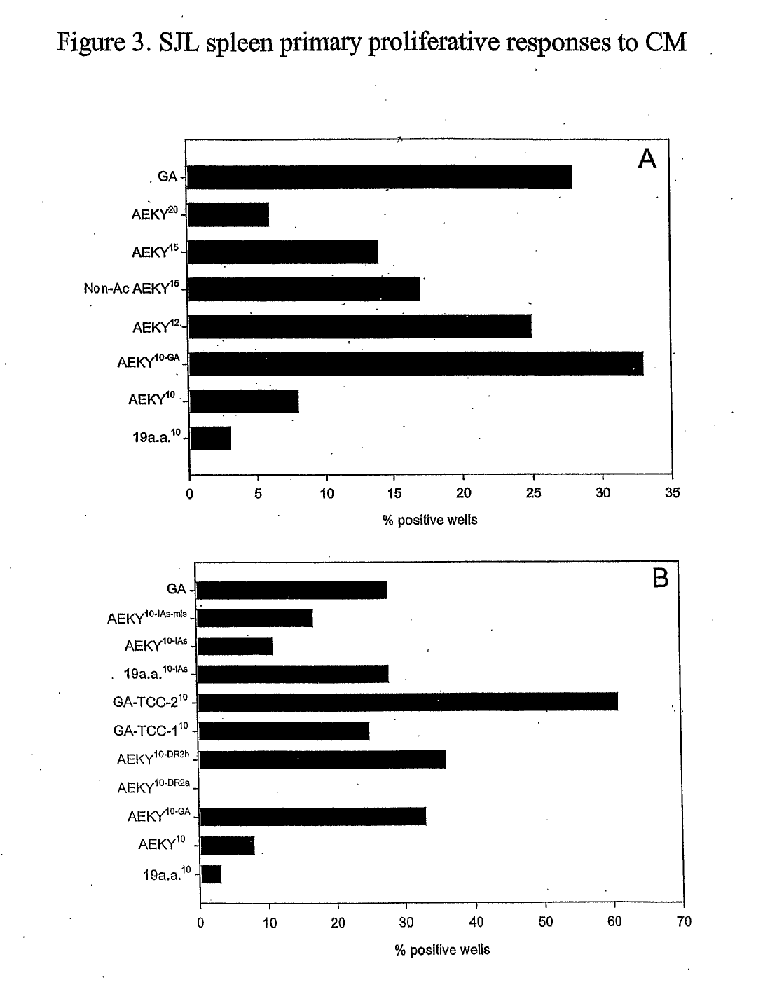

Complex mixtures (CM) were designed to assess the ability of small, defined peptide mixtures to stimulate PBMC and to test the influence of acetylation, peptide length and MHC anchor bias on these responses. The basic complex mixture with the greatest complexity is a 10-mer containing any of the 19 non-cysteine amino acids (cysteine is omitted to avoid the formation of secondary structures). Changes were based on this basic framework to characterize the influence of size and composition on stimulatory or therapeutic potential. As the number of amino acids (aa) in each position of the peptide decreases, so does the complexity of the peptide mixture. The immunogenicities of CM were assessed by measuring the proliferative responses of both healthy donor- and MS-derived PBMC upon primary and secondary exposure to antigens or in primary proliferations using mouse spleen cells. In preliminary experiments using cells from mice and healthy donors, all acetylated CM were found to be more stimulatory than their non-acetylated counterpart with the exception of the 15-mers (data not shown), and therefore studies were continued using only the acetylated 10-, 12-, and 20-mers. Both in healthy donors (FIG. 1A,C) and with mouse spleen cells (FIG. 3A), longer mixtures of 15 to 20 aa (linear or branched, data not shown) generated proliferative responses less frequently than shorter mixtures of 10 to 12 aa length. Subsequently, experiments focused on these shorter, more stimulatory CM in MS patients and also in therapeutic experiments in mice. In nine healthy donors (FIG. 1A-B) and five MS patients (FIG. 2A-B), primary and secondary proliferative responses were most frequently observed upon stimulation with the polypeptidic GA, and this was also true for mouse primary responses. However, the effectiveness of AEKY10-GA, AEKY12 and non-Ac-AEKY15 compared to the longer 20mers, albeit in a lower proportion of wells than GA, suggests that increasing the length of the mixture does not necessarily increase its stimulatory potential.

### Incorporating MHC Anchor Amino Acids, MHC Bias, Increases the Stimulatory Potential of CM

Using published data of known MHC anchors for the two HLA-DR2 alleles expressed in the HLA-DR15 haplotype, i.e. DRB1*1501 or DR2b, and DRB5*0101 or DR2a, AEKY-based CM were designed in order to test whether incorporation of MHC anchor aa would increase the stimulatory potential of these compounds. Compared to the basic AEKY10, AEKY10 containing either HLA-DR2a or HLA-DR2b anchors were significantly more stimulatory in both healthy donors and patients (FIG. 1C-D, 2A-B). While this result could be anticipated in HLA-DR2+ patients and HLA-DR2+ healthy donors, it was also observed in the non-DR2 donors (data not shown). Sharing of similar aa in specificity pockets of many HLA-DR alleles, e.g. in pocket 1, may be responsible for this observation.

In spleens from SJL mice, the introduction of key MHC anchors for IAs in appropriate pockets (AEKY10-IAs) or even out of alignment (AEKY10-IAs-mis) markedly enhanced the stimulatory potential of AEKY10 (FIG. 3B). The significant contribution of the IAs anchors was further emphasized by increased proliferation in response to random peptides (19 a.a.) once the anchors were introduced, even in the absence of the GA-derived aa bias. Interestingly, AEKY10-DR2b was also significantly more stimulatory than unbiased peptides (AEKY10) in mice.

### Glatiramer Acetate Bias is not Required for the Stimulatory Potential of CM

The stimulatory nature of GA has often been attributed to its unique composition. Accordingly, the AEKY10 at GA molar ratios generated stronger proliferative responses than the AEKY10 at equimolar ratios, which in turn was more stimulatory than the 19a.a.10. However, such GA molar bias was not absolutely required in the longer peptides as evident by the high frequency of responses to equimolar acetylated AEKY12 or non-acetylated AEKY15 particularly in secondary proliferations (FIGS. 1 and 2). In both healthy donors and MS patients, the MHC bias of AEKY10-DR2b was able to match and even surpass the increased stimulatory potential or AEKY10-GA compared to the AEKY10 at equimolar amounts; remarkably, this same DR2b-biased observation was made in the IAs SJL mouse. Additionally, introduction of the IAs MHC bias into the 19a.a.10 (FIG. 3A-B) creates a CM with equal stimulatory potency as AEKY10-GA and more stimulatory than AEKY10-IAs, again stressing that there is only a relative requirement for the aa composition of GA, and that better than GA-biased CM may conceivably be designed as well.

PSCL are a valuable tool to identify potential T cell antigens and were used with a GA-specific TCC to identify those aa at each position in a peptide or sets of peptides that are stimulatory for this clone. One mixture was designed containing all potentially stimulatory aa in each position (those PSCL inducing SIs 1.2 times greater than the average SI at each position-GA-TCC-2). Another, less complex mixture was created with presumably a higher activation threshold, composed only of the most stimulatory aa at each position (GA-TCC-1) and thus containing less sequences than GA-TCC-2. Both of these mixtures were highly stimulatory in patients and healthy donors (FIG. 1C-D, FIG. 2A-B), however GA-TCC-2 (the most complex mixture), was found to be more stimulatory overall. In mice this more complex peptide was also highly stimulatory (FIG. 3B), much more so than other CM or even GA. Notably, alanine, glutamic acid and lysine appear frequently in these GA-clone “designed” mixtures, yet their presence at defined positions in the linear peptide seemed to result in CM with sequences more stimulatory than the ones contained in AEKY10-GA.

### CM-specific T Cell Lines Exhibit Cross Reactivity to Myelin Proteins

The most conceivable mechanism of action for GA involves the ability to induce bystander suppression. Th2/Th3 immunosuppressive GA-specific T cells have been shown to recognize myelin proteins, and this cross-reactivity allows for the release of immunosuppressive factors at the site of myelin breakdown and thereby bystander suppression. CM-specific lines established from both healthy donors and patients, as well as from CM-immunized mice, were tested for their ability to recognize whole myelin proteins or peptides previously associated with CNS autoimmunity. Table 2 below shows a set of human T cell lines which demonstrated proliferative CM-specific cross-reactivity to myelin peptide pools. In most cases, CM-specific lines were most responsive to the original antigen used for generating the culture with often lower stimulation indices in response to myelin proteins/peptides. While the preliminary data in patients suggest a predominance of cross reactivities to MOBP or MBP peptides, this observation requires further study.

TABLE 2
Cross reactivity of human CM-specific T cell lines to myelin peptide pools

| | | | Cross | SI |
|---|---|---|---|---|
| | T cell line | | reactivity to | to myelin |
| Donor | specificity | SI to CM | myelin pool | pool |
| HD23677-9 | AEKY^{10-GA} | 2 | PLP | 3 |
| | AEKY^{10-DR2b} | 5 | PLP | 2 |
| | GA-TCC-2¹⁰ | 5 | PLP | 2 |
| MS patient C.H. | AEKY^{15 non-Ac} | 2 | PLP, MOBP, | 6, 3, 2.5 |
| | | | MBP | |
| | AEKY^{10-DR2a} | 36 | MBP | 3 |
| | AEKY^{10-DR2b} | 2 | MOBP | 2.5 |
| MS patient P.G. | AEKY^{15 non-Ac} | 4 | MOBP | 2 |
| | AEKY^{10-DR2a} | 21 | MOBP | 2 |
| | GA-TCC-2¹⁰ | 3 | MOBP | 2 |
| MS patient J.P. | AEKY^{15 non-Ac} | 25 | MBP | 2 |

T cell lines isolated from MS patients and healthy donors (HD) were used after primary and secondary proliferations to confirm the specificity of the line. Lines specific for several CM are shown with SI in proliferation assays compared to control wells in response to the initial immunizing CM and pools of myelin peptides at 20 μg/ml. Bulk myelin pools contained from 4 to 5 distinct peptides within each pool, as described in Materials and methods, each in equal amounts.

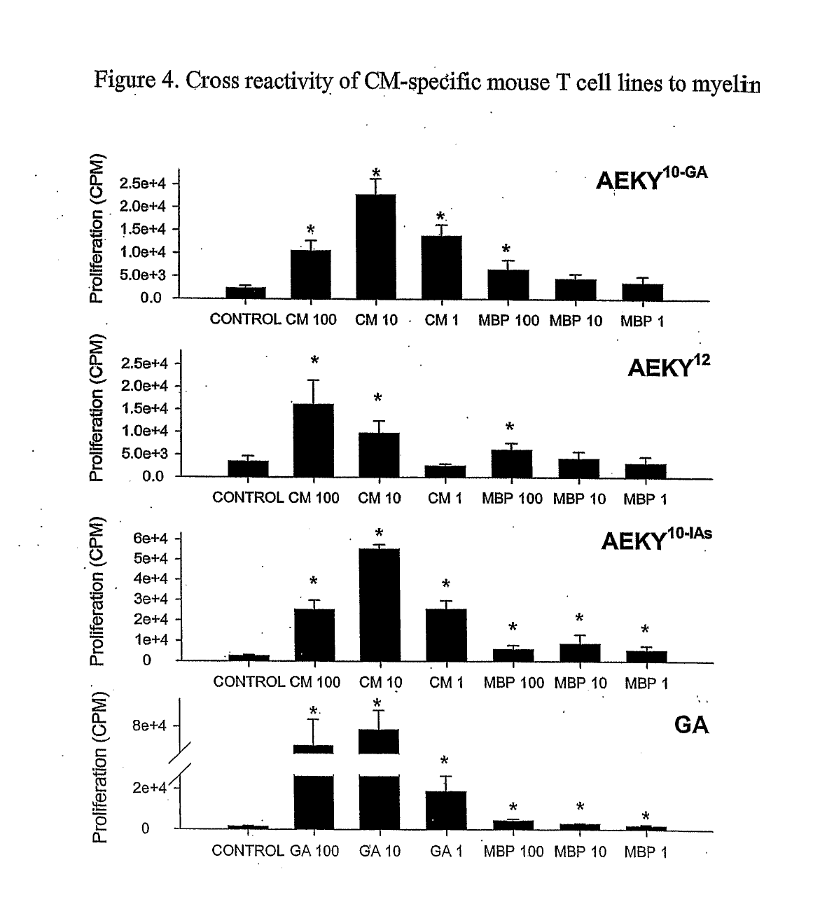

SJL mice were immunized with CM and spleen cells collected ten days later to enable the study of CM-specific T cells that might otherwise exist at very low frequencies. Cells from AEKY10-GA, AEKY12, AEKY10-IAs and also GA-immunized mice responded to their immunizing antigen with very high SIs, but also cross reacted with whole human MBP to varying degrees (FIG. 4). Responses of CFA only-immunized spleens to MBP were negligible (data not shown). Notably, cross reactivity to myelin proteins in both human and mouse CM-specific T cell lines was most often observed with those CM based on the AEKY10, -12 or -15 mixtures and found to be most stimulatory in proliferative experiments.

### Prophylactic Potential of CM in EAE

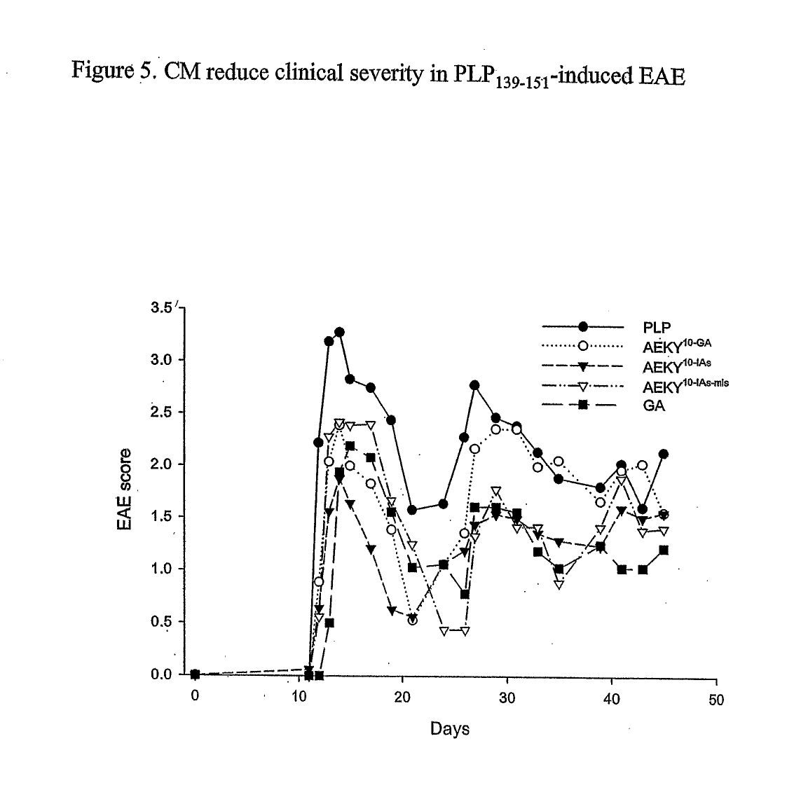

CM were delivered in a single dose along with PLP to test the prophylactic potential of these compounds to alter the course or severity of EAE in SJL mice. Each CM listed in Table 1 was tested at 500 μg added to the inoculum with PLP 139-151 and CFA and mice were monitored daily for signs of disease. At this single dose, AEKY10-GA and AEKY10-IAs reduced the cumulative disease score over the first bout of disease by approximately half (Table 3 below, FIG. 5), results similar to those obtained using GA. Interestingly the IAs bias of 19a.a.10-IAs, while stimulatory to mouse cells, was not effective prophylactically, suggesting a role for some component of AEKY in influencing prophylactic efficacy. Similar to their stimulatory potential, the prophylactic efficacy of the GA molar bias introduced in AEKY10-GA compared to AEKY10 could be duplicated by the introduction of the IAs anchor bias in AEKY10-IAs. Only AEKY10-IAs and GA were able to significantly reduce the cumulative disease course throughout the relapse phase, reducing scores by approximately one-third. While these compounds were among the most stimulatory in mouse proliferative experiments, other highly stimulatory mixtures including AEKY12 or GA-TCC-210 had no effects on the disease course at this dose (data not shown).

TABLE 3
Summary of CM prophylaxis in EAE experiments

| | Cumulative | Cumulative |
|---|---|---|
| | disease score | disease score |
| Treatment | (day 0-24) | (day 24-45) |
| PLP | 19.3 ± 1.4 | 21.4 ± 2.0 |
| PLP + AEKY¹⁰ | 19.7 ± 2.8 | 18.3 ± 3.6 |
| PLP + AEKY^{10-GA} | 11.8 ± 1.4* | 19.1 ± 2.7 |
| PLP + 19 a.a.^{10-IAs} | 14.9 ± 2.2 | 18.3 ± 3.9 |
| PLP + AEKY^{10-IAs} | 9.5 ± 1.4** | 13.8 ± 2.1* |
| PLP + AEKY^{10-IAs-mis} | 13.4 ± 1.8* | 16.2 ± 2.5 |
| GA | 10.2 ± 1.6** | 13.5 ± 3.0* |

Data are means ± SEM.
The cumulative score represents the mean of the summation of single scores recorded in each mouse during the initial disease course (days 0-24) and the remaining disease course (days 24-45).
Data represents the results from 2 pooled experiments with greater than 10 mice per group.
*P < 0.05 when compared to PLP immunized mice.
**P < 0.001 when compared to PLP immunized mice.

Optimization of Mixture Formulas with PSCL-Related Techniques

Using a variation of our positional scanning technology, two of the complex mixtures from Table 1 (also below) are optimized. AEKY10-IAs is less complex (a mixture of 294,912 peptides) as compared to 19a.a.10-IAs, a highly complex mixture (a mixture of 3,387,303,432 peptides).

| | | P1 | P2 | P3 | P4 | P5 | P6 | P7 | P8 | P9 | P10 |
|---|---|---|---|---|---|---|---|---|---|---|---|
| AEKY^{10-IAs} | Ac- | AEKY | AEKY | AEKY | AEKY | KHRV | AEKY | ILV | HRK | AEKY | PI |
| 19a.a.^{10-IAs} | Ac- | X | X | X | X | KHRV | X | ILV | HRK | X | PI |

X = equimolar amount of all 20 L amino acids excluding Cysteine

In order to optimize these CMs, position 5 (involved in TCR binding) and position 1 or 4 (involved in MHC binding) are defined in a manner similar to how positional scanning is usually performed. For example, for complex mixture AEKY10-IAs, 40 mixtures are made, 20 with Position 5 defined with one of the 20 amino acids (Samples 1-20) and 20 with position 1 or 4 defined (samples 21-40). Sample formulas are shown below in Table 4. In other experiments, substitutions will be made at positions not known to play a role in binding.

TABLE 4
Formulas for optimization of complex mixtures experiment

| AEKY^{10-IAs} | | P1 | P2 | P3 | P4 | P5 | P6 | P7 | P8 | P9 | P10 |
|---|---|---|---|---|---|---|---|---|---|---|---|
| Original | Ac- | AEKY | AEKY | AEKY | AEKY | KHRV | AEKY | ILV | HRK | AEKY | PI |
| Sample-1 | Ac- | AEKY | AEKY | AEKY | AEKY | A | AEKY | ILV | HRK | AEKY | PI |
| Sample-2 | Ac- | AEKY | AEKY | AEKY | AEKY | C | AEKY | ILV | HRK | AEKY | PI |
| Sample-3 | Ac- | AEKY | AEKY | AEKY | AEKY | D | AEKY | ILV | HRK | AEKY | PI |
| Sample-4 | Ac- | AEKY | AEKY | AEKY | AEKY | E | AEKY | ILV | HRK | AEKY | PI |
| Sample-5 | Ac- | AEKY | AEKY | AEKY | AEKY | F | AEKY | ILV | HRK | AEKY | PI |
| Sample-6 | Ac- | AEKY | AEKY | AEKY | AEKY | G | AEKY | ILV | HRK | AEKY | PI |
| Sample-7 | Ac- | AEKY | AEKY | AEKY | AEKY | H | AEKY | ILV | HRK | AEKY | PI |
| Sample-8 | Ac- | AEKY | AEKY | AEKY | AEKY | I | AEKY | ILV | HRK | AEKY | PI |
| Sample-9 | Ac- | AEKY | AEKY | AEKY | AEKY | K | AEKY | ILV | HRK | AEKY | PI |
| Sample-10 | Ac- | AEKY | AEKY | AEKY | AEKY | L | AEKY | ILV | HRK | AEKY | PI |
| Sample-11 | Ac- | AEKY | AEKY | AEKY | AEKY | M | AEKY | ILV | HRK | AEKY | PI |
| Sample-12 | Ac- | AEKY | AEKY | AEKY | AEKY | N | AEKY | ILV | HRK | AEKY | PI |
| Sample-13 | Ac- | AEKY | AEKY | AEKY | AEKY | P | AEKY | ILV | HRK | AEKY | PI |
| Sample-14 | Ac- | AEKY | AEKY | AEKY | AEKY | Q | AEKY | ILV | HRK | AEKY | PI |
| Sample-15 | Ac- | AEKY | AEKY | AEKY | AEKY | R | AEKY | ILV | HRK | AEKY | PI |
| Sample-16 | Ac- | AEKY | AEKY | AEKY | AEKY | S | AEKY | ILV | HRK | AEKY | PI |
| Sample-17 | Ac- | AEKY | AEKY | AEKY | AEKY | T | AEKY | ILV | HRK | AEKY | PI |
| Sample-18 | Ac- | AEKY | AEKY | AEKY | AEKY | V | AEKY | ILV | HRK | AEKY | PI |
| Sample-19 | Ac- | AEKY | AEKY | AEKY | AEKY | W | AEKY | ILV | HRK | AEKY | PI |
| Sample-20 | Ac- | AEKY | AEKY | AEKY | AEKY | Y | AEKY | ILV | HRK | AEKY | PI |
| Sample-21 | Ac- | A | AEKY | AEKY | AEKY | KHRV | AEKY | ILV | HRK | AEKY | PI |
| Sample-22 | Ac- | C | AEKY | AEKY | AEKY | KHRV | AEKY | ILV | HRK | AEKY | PI |
| . . . | | | | | | | | | | | |
| Sample-40 | Ac- | Y | AEKY | AEKY | AEKY | KHRV | AEKY | ILV | HRK | AEKY | PI |

By screening these 40 samples (for complex mixture AEKY10-IAs) in the EAE model and comparing their activity to the original CM, amino acids are identified for inclusion in positions 1 and 5. These modified complex mixtures are made and screened in the EAE model to confirm their activity. The results may be used to create complex peptide mixture formulas with alternative amino acids residue groups for inclusion in positions 1 and 5, to increase the activity of the mixtures.

This experiment is also done for a complex mixture of more complexity such as 19a.a.10-IAs (it can be done in parallel with the other complex mixture). By modifying the two types of complex mixtures (highly complex and less complex), the contribution of complexity to the activity of complex mixtures in the EAE model can be established and used to design mixtures with higher levels of activity.

These experiments involve 40 samples for each CM for a total of 80 samples. Each of these samples is run in 10 different mice EAE models (800 mice plus controls) for approximately two months. Based on the data gathered in these experiments, one to three new complex mixtures derived from each of the original complex mixtures are synthesized, for a total of 6 new samples, each to be screened in 10 different mice EAE models (60 mice plus controls) for approximately two months.

Complex mixture libraries can also be screened using other experimental methods. For example, PBMCs can be isolated from blood drawn from MS patients or from healthy donors. These cells can then be used in experiments that measure the level of cellular activation or proliferation after exposure to various mixtures of peptides. Complex mixture peptide formulas can be generated with reduced or increased levels of complexity based on the results of these experiments. In some embodiments, two or more rounds of complex peptide mixture reformulations take place, with the possible choices of the amino acids in certain positions within the peptides of the mixture becoming more and more limited. In some embodiments, the screening of mixtures results in the selection of a precise formula for a particular peptide.

Other screening methods that can be used with the invention include other animal-based disease models. For example, the effects of complex peptide mixtures on the cardiovascular systems of laboratory animals can be measured after exposing the animals to the mixtures. Other in vivo screening models include models of anxiety and depression which are well-known in the art, such as: light/dark choice, which is designed to measure an animal's tendency to stay in dark; the elevated plus maze which is designed to measure an animals' response to stimulation designed to enhance fear and anxiety; stress-induced hyperthermia which is designed to measure core body temp of animal under induced hyperthermia; fear potentiated startle, which is designed to measure response to loud noise that has previously been paired with electric shock. Other screening models include obesity studies which are designed to measure food or calorie intake. Screening models of cognition include the Morris Water Maze, which is designed to measure the animal's ability to learn to find hidden escapes based on learned clues. Screening models of neurological or motor disorders include test of grip strength of the animal's fore and hind limbs, and the rotarod which is used to access motor coordination and balance. Other screening models include models of pain, including tail flick, hot plate and plaster and writhing. Other in vivo screening models are well-know to those in the art, and can be used to screen the complex peptide mixtures.

Based on the effects of particular mixtures, new mixtures of reduced or increased diversity can be formulated.

### Conclusions:

Shorter complex peptide mixtures were created in order to test the ability of a less complex, and more structurally defined mixture to exert both stimulatory and ultimately therapeutic properties relevant to autoimmune processes. Primarily, the goal is to develop mixtures of limited complexity with the intention to study subtle changes in basic factors including peptide length, structure or composition and their relationships to stimulatory and therapeutic potentials. In some cases, the stimulatory potential of peptides of different lengths suggested that in the 10 to 20 amino acid range, shorter peptides of 10-12 amino acids, particularly acetylated molecules, were far more stimulatory to bulk T cells from healthy donors, MS patients, and SJL mice. Interestingly, the increased presence of A and K relative to E and Y in AEKY^{10-GA} was more stimulatory than those aa at equimolar ratios in AEKY¹⁰. The hydrophobicity and size of Y with its enrichment in AEKY¹⁰ may account for relatively reduced binding and thus lower stimulatory potential. While numerous reports suggest that the large size of GA is ultimately responsible for its strong stimulatory potential, we found reasonable stimulatory indices using smaller peptides, both in mice and men.

The most marked increases in CM stimulatory potential occurred when an MHC bias was introduced into the peptide AEKY¹⁰ or into the 19a.a.¹⁰. While binding studies for these CM remain to be carried out, the data reflect that DR2a or DR2b anchor residues enhance the stimulatory potential of these 10mers. There was no significant difference between increases attributable to DR2a versus DR2b bias in patients or healthy donors, however, the DR2b-biased AEKY^{10-DR2b} was found to be far more stimulatory in IA^{s} mice populations whereas responses to DR2a-biased CM were negligible. These data suggest that the MHC bias introduced may either by virtue of its aa composition or the sharing of common anchors result in cross-species efficacy. In referring to Table 1, one sees that I, L and V are shared as the DR2b and IA^{s} anchor residues at P7, and this might explain the increased stimulatory potential of DR2b-biased CM in SJL mice compared to DR2a-biased CM and even IA^{s}-biased CM. Currently, we do not know, however, whether these speculations are in fact correct, particularly since the HLA/MHC-class II binding groove is open at either end, and binding of peptides may occur in multiple registers and with specific aa in different pockets.

Studies involving GA-specific TCC and PSCL served to identify key residues driving the stimulatory properties of GA and specify the “ideal antigen” component in GA that is recognized by GA-specific clones. Mixtures that contain more aa at each position and are thus more complex (GA-TCC-2, as compared to GA-TCC-1) were more stimulatory throughout studies in healthy donors (of all HLA types, not only DR2+), patients and mouse cells.

Although substantial consideration is given to the ability of GA to compete for peptide binding as a mechanism of action, it seems unlikely that this property is operative in vivo, since large quantities of GA would be required to mediate these effects, and these will probably never be reached. Bystander suppression is a more attractive immunomodulatory mechanism, and several groups have recently demonstrated that bystander suppression indeed occurs in GA-treated patients (Duda et al., 2000; Gran et al., 2000; Neuhaus et al., 2000; Chen et al., 2001). Cross reactivity with autoantigenic peptides is conceptionally an important prerequisite for bystander suppression.

The majority of CM developed using MHC bias or PSCL-driven data were highly stimulatory and thus took advantage of the degeneracy of TCR recognition. However, the stimulatory capacity of a CM did not translate directly into efficacy in reducing the cumulative severity of EAE. In fact, only IA^{s}-biased 10mers matched the reduction in disease observed with GA or GA-molar ratio-biased 10mers AEKY^{10-GA}. Coimmunizaion with GA-TCC-2¹⁰ (lower threshold aa in peptides) or AEKY^{10-DR2b}, while both very stimulatory in culture, had no effects on the disease course at these doses. Moreover, only GA and AEKY^{10-IAs} were able to significantly reduce the overall disease score throughout the first and subsequent disease exacerbations. The effectiveness of these molecules at reducing disease severity when introduced simultaneously with encephalitogen suggests that this property could be due to mere competition for binding between the encephalitogenic PLP peptide and the co-immunizing CM.

In summary, the present invention describes the preparation of short, peptidic complex mixtures that act as potent multi-antigens and stimulate large numbers of T cells in patients and healthy individuals. These peptide mixtures generated T cell lines with significant cross reactivity to myelin proteins and peptides, in addition to effectively reducing the cumulative disease score in EAE by half. The present invention also includes the use of GA-specific clones to identify the contributions of individual aa to the overall value of these peptides, preferably across multiple species. The simple, well-defined composition of these CM makes them a valuable tool to study the basic factors that can moderate mixture efficacy, by allowing for even the most subtle changes in structure or composition. The present invention suggests that short peptidic mixtures are an ideal tool to exploit T cell degeneracy and act as the platform for future vaccine or drug design on which to investigate and build immunotherapeutic molecules that can target a broad population with autoimmune diseases, cancer or exploit them for vaccine design in infectious diseases.

1-84. (canceled)

85. A complex peptide mixture, comprising a plurality of peptides having a length within the range of 8 to 20 amino acids, wherein said mixture comprises peptides having a degree of diversity at defined positions in the peptide chain, wherein the degree of diversity in at least one defined position is different from the degree of diversity in at least one other defined position, and wherein in a majority of the mixture, the peptides include in at least four positions all of A, E, K, and Y and no other amino acids.

86. The peptide mixture of claim 85, wherein in at least one position, a majority of the peptides in the mixture include each of one or two amino acids and no other amino acids.

87. The peptide mixture of claim 85, wherein the amino acids in said at least four positions include all of A, E, K, and Y and no other amino acids in at least 75% of the peptides in the mixture.

88. The peptide mixture of claim 85, wherein the amino acids in said at least four positions include all of A, E, K, and Y and no other amino acids in at least 90% of the peptides in the mixture.

89. The peptide mixture of claim 85, wherein the amino acids in said at least four positions include all of A, E, K, and Y and no other amino acids in at least 95% of the peptides in the mixture.

90. The peptide mixture of claim 85, wherein the amino acids in said at least four positions include all of A, E, K, and Y and no other amino acids in substantially all of the peptides in the mixture.

91. The peptide mixture of claim 85, wherein a majority of the peptides in the mixture are acetylated at the N-terminal amino acid.

92. The peptide mixture of claim 85, wherein the amino acids A, E, K, and Y in said at least four positions are present in the following molar proportions: about 6:about 2:about 5:about 1.

93. The peptide mixture of claim 85, wherein in at least one position, a majority of the mixture consists of peptides that include all of I, L, and V and no other amino acids.

94. The peptide mixture of claim 85, wherein in at least one position, a majority of the mixture consists of peptides that include all of H, R, and K and no other amino acids.

95. The peptide mixture of claim 85, wherein in at least one position, a majority of the mixture consists of peptides that include all of K, H, R, and V and no other amino acids.

96. A complex peptide mixture, comprising a plurality of peptides having a length within the range of 8 to 20 amino acids, wherein said mixture comprises peptides having a degree of diversity at defined positions in the peptide chain and wherein at least in a majority of the mixture, the amino acids in the peptides include all of the following specified amino acids and no other amino acids:
in at least four positions: A, E, K, and Y;
in at least one position: I, L, and V;
in at least one position: H, R, and K; and
in at least one position: P and I.

97. The peptide mixture of claim 96, wherein position P1 is the N-terminal residue.

98. The peptide mixture of claim 96, wherein the N-terminal residue is acetylated.

99. The peptide mixture of claim 96, wherein A, E, K, and Y are present in at least four of positions P1, P2, P3, P4, P6, and (if present), P9.

100. The peptide mixture of claim 96, wherein said peptides have a length of at least 10 amino acids and A, E, K, and Y are present in all of positions P1, P2, P3, P4, P6, and P9.

101. The peptide mixture of claim 96, wherein at least in a majority of the mixture, the amino acids at the carboxy terminus of the peptides include both P and I and no other amino acids.

102. The peptide mixture of claim 96, wherein a majority of the peptides in the mixture have, in at least one position, the same amino acid.

103. A method of treating a disease, comprising administering to a vertebrate a complex peptide mixture, wherein said mixture comprises peptides having a constrained degree of diversity at each of at least 4 defined positions in the peptide chain and wherein the constrained degree of diversity in at least one defined position is different from the constrained degree of diversity in at least one other defined position.

104. The method of claim 103, wherein said disease is selected from the group consisting of multiple sclerosis and experimental autoimmune encephalomyelitis.

105. A method for stimulating an immune cell comprising administration of a complex peptide mixture to said cell, wherein said mixture comprises peptides having a constrained degree of diversity at each of at least 4 defined positions in the peptide chain, and wherein the degree of diversity in at least one defined position is different from the degree of diversity in at least one other defined position.

106. The method of claim 105, wherein said stimulating of an immune cell occurs in vivo.

107. The method of claim 105, wherein said administering step is selected from the group consisting of intravenous delivery, intramuscular delivery, delivery via the gastrointestinal tract, and transdermal delivery.

108. A method for suppression of an immune reaction to an antigen, comprising administering a complex peptide mixture to an individual, said complex peptide mixture comprising peptides having a constrained degree of diversity at each of at least 10 defined positions in the peptide chain, and wherein the degree of diversity in at least one or two defined positions is different from the degree of diversity in at least one or two other defined positions.

109. The method of claim 108, wherein said immune reaction is an autoimmune reaction.

110. The method of claim 109, wherein said antigen is derived from myelin.

111. The method of claim 108, wherein said peptide complex mixture comprises peptides of a defined length and formula comprising at least one position with a specific amino acid and at least one position with two to six possible amino acids.

112. The method of claim 108, wherein said peptide complex mixture is a less-diverse subset of a complex mixture having a higher degree of diversity.

113. The method of claim 108, wherein said complex mixture with a higher degree of diversity is a mixture whose formula is selected from the group consisting of the formulas listed in Table 1.

114. The method of claim 108, wherein the activity of said peptide complex mixture is evaluated by an assay selected from the group consisting of a proliferation assay, a cytokine assay and a ⁵¹Cr release assay.

115. The method of claim 110, wherein said suppression of an autoimmune reaction to myelin proteins is due to a mechanism selected from the group consisting of MHC blockade, TCR antagonism, tolerance induction, immune deviation/bystander suppression and cross reactivity with an antigen derived from a protein expressed within the central nervous system.

116. The method of claim 110, wherein said myelin antigen is derived from a protein selected from the group consisting of myelin basic protein, proteolipid protein and myelin oligodendrocyte glycoprotein.

117. The method of claim 108, wherein said antigen is derived from a protein expressed by a cell found in the central nervous system.

118. A method for inducing an anti-inflammatory response from immune system cells comprising administering a complex peptide mixture to immune system cells, said complex peptide mixture comprising peptides having a constrained degree of diversity at each of at least 10 defined positions in the peptide chain, and wherein the constrained degree of diversity in at least one or two said defined positions is different from the degree of diversity in at least another defined position.

119. The method of claim 118, wherein said anti-inflammatory response comprises an alteration in immune cell activity selected from the group consisting of an upregulation of Th2/Th3 cell activity and a downregulation of Th1 cell activity.

120. The method of claim 118, wherein said anti-inflammatory response comprises an increase in the release of cytokines by cells, said cytokines selected from the group consisting of IL-4, IL-5, IL-10, TGF-beta and IL-13.

121. A method for creating a high affinity peptide ligand of a defined formula for a receptor of a T cell that is reactive to a myelin antigen, copolymer or an active mixture, comprising the deconvolution of a complex peptide mixture of a formula selected from the group consisting of the formulas listed in Table 1.

122. A complex peptide mixture, wherein said mixture comprises peptides between 4 and 100 amino acid residues in length, wherein said mixture has a constrained degree of diversity at each of at least 10 defined positions in the peptide chain, and wherein the degree of diversity in at least one defined position is different from the degree of diversity in at least one other defined position.

123. The complex peptide mixture of claim 122, wherein the constrained degree of diversity is created by limiting the possible amino acids at each of the said defined positions to a list of possible amino acids that includes less than 10 amino acids.

124. The complex peptide mixture of claim 123, wherein the list of possible amino acids for at least two of the said defined positions includes only 1, 2, or 3 amino acids.

125. The complex peptide mixture of claim 122, wherein a majority of the peptides in the mixture have a length between about 4 and about 30 amino acid residues.

126. The complex peptide mixture of claim 122, wherein a majority of the peptides in the mixture vary in length by no more than 4 amino acid residues.

127. The complex mixture of claim 122, wherein a majority of the peptides in the mixture are acetylated.

128. The complex mixture of claim 122, wherein at least one position of one peptide in the mixture is occupied by a D-amino acid.

129. A method of enhancing a biological property of a complex mixture of peptides, peptidomimetics, or peptides and peptidomimetics, comprising:
providing a set of complex mixtures of reduced complexity that have a reduced degree of diversity from said complex mixture at least at one position;
testing each of said complex mixtures of reduced complexity and said complex mixture in an assay;
identifying complex mixtures of reduced complexity that have a greater or lesser activity in said assay than said complex mixture; and
enhancing the biological property of said complex mixture by incorporating the complexity-reducing features of at least one of said complex mixtures of reduced complexity that have greater or lesser activity in said assay than said complex mixture into the formula of said complex peptide mixture.

130. The method of claim 129, wherein said biological property is selected from the group consisting of the ability to stimulate an immune system activity and the ability to suppress an immune system activity.

131. The method of claim 130, wherein said immune system activity is selected from the group consisting of the clonal expansion of an immune system cell, the differentiation of an immune system cell, the activation of an immune system cell, the creation of an anergic state in an immune system cell, the creation of a memory immune cell population and the secretion of cytokines from an immune system cell.

132. The method of claim 129, wherein said complex mixtures of reduced complexity comprise mixtures where the identity of the residue or amino acid in one or more positions in the mixture formula is limited to one functionality or amino acid.

133. The method of claim 129, wherein said complex mixtures of reduced complexity comprise mixtures wherein the identity of the residue or amino acid in one or more positions in the mixture formula is limited to a formula less diverse than the formula for that position in said complex mixture of peptides, peptidomimetics, or peptides and peptidomimetics.

134. The method of claim 129, wherein said assay is selected from the group consisting of an in vitro assay of peptide, peptidomimetic, or peptides and peptidomimetics recognition by an immune cell population and an assay of the effects of mixture administration on an organism.

135. The method of claim 134, wherein said assay of the effects of mixture administration on an organism is an assay of disease progression in the EAE mouse model.

136. The method of claim 129, wherein enhancing the biological activity of said complex mixture of peptides, peptidomimetics, or peptides and peptidomimetics comprises the limiting of the identity of the amino acid or residue at a position in the compound formula to a single amino acid or functionality.

137. The method of claim 129, wherein said biological property is the response measured in an in vivo model of disease.

138. The method of claim 137, wherein said in vivo model is selected from the group comprising a model of anxiety, depression, obesity, cognition, neurological disorder, motor disorder, or pain.

139. A complex peptide mixture, comprising a plurality of peptides having a length within the range of 8 to 20 amino acids, wherein said mixture comprises peptides having a degree of diversity at defined positions in the peptide chain, and wherein at least in a majority of the mixture, the identity of 10 contiguous amino acids in the peptides are defined by the following formulas: FW-EF-EK-AEK-AKY-ANY-ANY-AINV-ASV-Y; or EFWY-EFIVWY-EFKQ-AEKQ-AKQY-ANQY-AGNSY-AGINSV-AIQSV-IKRSVY.

140. The peptide mixture of claim 139, wherein position P1 of the 9 contiguous amino acid residues is the N-terminal peptide.

141. A complex peptide mixture, comprising a plurality of peptides having a length within the range of 8 to 20 amino acids, wherein said mixture comprises peptides having a degree of diversity at defined positions in the peptide chain, wherein the degree of diversity in at least one defined position is different from the degree of diversity in at least one other defined position, wherein in a majority of the mixture, the peptides include in at least one but no more than five positions all of A, E, K, and Y and no other amino acids.
